(19) United States
(12) Patent Application Publication
(10) Pub. No.: US 2025/0302426 A1
(43) Pub. Date: Oct. 2, 2025

(54) CONTINUOUS NONINVASIVE BLOOD PRESSURE MEASUREMENT

(71) Applicant: Masimo Corporation, Irvine, CA (US)

(72) Inventors: Taewoo Ha, Irvine, CA (US); Benjamin Rumbaugh, Mission Viejo, CA (US); David Dalke, Rancho Santa Margarita, CA (US); Valery G. Telfort, Irvine, CA (US)

(21) Appl. No.: 19/092,862

(22) Filed: Mar. 27, 2025

Related U.S. Application Data
(60) Provisional application No. 63/571,357, filed on Mar. 28, 2024.
(60) Provisional application No. 63/632,455, filed on Apr. 10, 2024.

(51) Int. Cl. A61B 8/04; A61B 8/00; H02K 33/18

(52) U.S. Cl. CPC A61B 8/04; A61B 8/4236; H02K 33/18; A61B 2562/0219

(57) ABSTRACT

A blood pressure monitoring system, comprising: an exciter configured to produce an acoustic signal, the exciter being provided on a first substrate portion; a detector spaced apart from the exciter, the detector being configured to detect the acoustic signal and to produce an electrical output signal, the detector being provided on a second substrate portion that is mechanically or acoustically decoupled from the first substrate portion, wherein the detector comprises at least four detectors; and a processor configured to determine a blood pressure measurement from the electrical output signal.

### INCORPORATION BY REFERENCE TO ANY PRIORITY APPLICATIONS

The present application claims priority benefit to U.S. Provisional Application No. 63/571,357 filed Mar. 28, 2024, entitled “CONTINUOUS NONINVASIVE BLOOD PRESSURE MEASUREMENT” and U.S. Provisional Application No. 63/632,455 filed Apr. 10, 2024, entitled “CONTINUOUS NONINVASIVE BLOOD PRESSURE MEASUREMENT,” each of which are hereby incorporated by reference herein in their entirety. Any and all applications for which a foreign or domestic priority claim is identified in the Application Data Sheet as filed with the present application are hereby incorporated by reference under 37 CFR 1.57 and made a part of this specification.

### BACKGROUND

### 1. Field

The field of this disclosure relates to devices and techniques for noninvasively measuring blood pressure.

### 2. Description of the Related Art

The human cardiovascular system is made up of the heart, blood vessels, and blood. The heart pumps blood through the blood vessels to transport oxygen, nutrients, etc., throughout the body.

Blood pressure is a measure of the pressure exerted by the circulating blood on the walls of the blood vessels and is typically measured in one of the large arteries. Blood pressure varies during the cardiac cycle from one heartbeat to the next. When the heart contracts, blood pressure momentarily rises and then subsequently falls until the next heartbeat. The systolic pressure is the maximum blood pressure attained during a cardiac cycle, while the diastolic pressure is the minimum blood pressure during the cardiac cycle. The mean arterial pressure (MAP) is the average blood pressure during the cardiac cycle. Blood pressure depends on a number of factors, including blood volume, cardiac output, vascular resistance, arterial stiffness, etc.

In medicine, blood pressure is a vital sign which can be used as an indicator of a patient's condition. Improved devices and techniques for measuring blood pressure can therefore help improve patient monitoring capabilities. A sensor is described in U.S. Patent Application Publication No. 2023/0284916 entitled “CONTINUOUS NONINVASIVE BLOOD PRESSURE MEASUREMENT” which is assigned to Masimo Corporation, Irvine, CA, and is incorporated by reference herein in its entirety.

### SUMMARY

In some aspects, the techniques described herein relate to a blood pressure monitoring system, including: an exciter configured to produce an acoustic signal, the exciter being provided on a first substrate portion; a detector spaced apart from the exciter, the detector being configured to detect the acoustic signal and to produce an electrical output signal, the detector being provided on a second substrate portion that is mechanically or acoustically decoupled from the first substrate portion, wherein the detector includes at least four detectors; and a processor configured to determine a blood pressure measurement from the electrical output signal.

In some aspects, the techniques described herein relate to a blood pressure monitoring system, wherein the at least four detectors are separated by at least one gap.

In some aspects, the techniques described herein relate to a blood pressure monitoring system, wherein the detector includes five detectors.

In some aspects, the techniques described herein relate to a blood pressure monitoring system, further including a memory device.

In some aspects, the techniques described herein relate to a blood pressure monitoring system, further including an inertial measurement unit (IMU).

In some aspects, the techniques described herein relate to a blood pressure monitoring system, wherein the first substrate portion and the second substrate portion are separated by a gap.

In some aspects, the techniques described herein relate to a blood pressure monitoring system, wherein the first substrate portion and the second substrate portion are separated by acoustically absorptive material.

In some aspects, the techniques described herein relate to a blood pressure monitoring system, wherein the processor is configured to determine the blood pressure measurement based on a measured phase delay between an electrical input signal and the electrical output signal.

In some aspects, the techniques described herein relate to a blood pressure monitoring system, wherein the first substrate portion and the second substrate portion are flexible.

In some aspects, the techniques described herein relate to a blood pressure monitoring system, further including a flexible circuit that connects the exciter and the detector to an electrical connector.

In some aspects, the techniques described herein relate to a blood pressure monitoring system, wherein the exciter and the detector include a piezo device or a microelectromechanical system.

In some aspects, the techniques described herein relate to a blood pressure monitoring system, further including an attachment element configured to attach the exciter and the detector to a forearm of a patient over a radial artery.

In some aspects, the techniques described herein relate to a blood pressure monitoring system, wherein the attachment element includes an adhesive substrate.

In some aspects, the techniques described herein relate to a blood pressure monitoring system, further including an alignment indicator to align a measurement axis of the exciter and the detector to the radial artery.

In some aspects, the techniques described herein relate to a blood pressure monitoring system, further including an attachment element configured to attach the exciter and the detector to a palm side of a wrist on a forearm of a patient.

In some aspects, the techniques described herein relate to a blood pressure monitoring system, further including an attachment element configured to attach the exciter and the detector to a patient, wherein the exciter is closer to a hand of the patient than the detector.

In some aspects, the techniques described herein relate to a blood pressure monitoring system, further including an attachment element configured to attach the exciter and the detector to a patient, wherein the detector is closer to a hand of the patient than the exciter.

In some aspects, the techniques described herein relate to a blood pressure monitoring system, further including an attachment element configured to attach the exciter and the detector to a patient, wherein the exciter and the detector are attached parallel to a length of an arm of the patient from an elbow to a wrist of the patient.

In some aspects, the techniques described herein relate to a blood pressure monitoring system, further including an attachment element configured to attach the exciter and the detector to a patient, wherein the exciter is positioned over a radial artery of the patient.

In some aspects, the techniques described herein relate to a blood pressure monitoring system, further including an attachment element configured to attach the exciter and the detector to a patient, wherein the detector is positioned over a radial artery of the patient.

In some aspects, the techniques described herein relate to a blood pressure monitoring system, including: an exciter configured to produce an acoustic signal, the exciter being provided on a substrate; a detector spaced apart from the exciter, the detector being configured to detect the acoustic signal and to produce an electrical output signal, the detector being provided on the substrate, wherein the detector includes at least four detectors; and a processor configured to determine a blood pressure measurement from the electrical output signal, wherein a gap in the substrate is along a line path from the exciter to the detector.

In some aspects, the techniques described herein relate to a blood pressure monitoring system, wherein the line path is in a straight line centered on the exciter from the exciter to the detector.

In some aspects, the techniques described herein relate to a blood pressure monitoring system, wherein the line path is off center from the exciter to the detector.

In some aspects, the techniques described herein relate to a blood pressure monitoring system, further including a memory device.

In some aspects, the techniques described herein relate to a blood pressure monitoring system, further including an inertial measurement unit (IMU).

In some aspects, the techniques described herein relate to a blood pressure monitoring system, wherein a path from the exciter to the detector via the substrate is longer than the line path from the exciter to the detector.

In some aspects, the techniques described herein relate to a blood pressure monitoring system, wherein the path from the exciter to the detector via the substrate is at least two times longer than a distance between the exciter and the detector.

In some aspects, the techniques described herein relate to a blood pressure monitoring system, wherein the path from the exciter to the detector via the substrate is at least five times longer than a distance between the exciter and the detector.

In some aspects, the techniques described herein relate to a blood pressure monitoring system, wherein the path from the exciter to the detector via the substrate includes acoustically absorptive material.

In some aspects, the techniques described herein relate to a blood pressure monitoring system, wherein the processor is configured to determine the blood pressure measurement based on a measured phase delay between an electrical input signal and the electrical output signal.

In some aspects, the techniques described herein relate to a blood pressure monitoring system, wherein the exciter and the detector include a piezo device or a microelectromechanical system.

In some aspects, the techniques described herein relate to a blood pressure monitoring system, further including an attachment element configured to attach the exciter and the detector to a forearm of a patient over a radial artery.

In some aspects, the techniques described herein relate to a blood pressure monitoring system, wherein the attachment element includes an adhesive substrate.

In some aspects, the techniques described herein relate to a blood pressure monitoring system, further including an alignment indicator to align a measurement axis of the exciter and the detector to the radial artery.

In some aspects, the techniques described herein relate to a blood pressure monitoring system, further including an attachment element configured to attach the exciter and the detector to a palm side of a wrist on a forearm of a patient.

In some aspects, the techniques described herein relate to a blood pressure monitoring system, further including an attachment element configured to attach the exciter and the detector to a patient, wherein the exciter is closer to a hand of the patient than the detector.

In some aspects, the techniques described herein relate to a blood pressure monitoring system, further including an attachment element configured to attach the exciter and the detector to a patient, wherein the detector is closer to a hand of the patient than the exciter.

In some aspects, the techniques described herein relate to a blood pressure monitoring system, further including an attachment element configured to attach the exciter and the detector to a patient, wherein the exciter and the detector are attached parallel to a length of an arm of the patient from an elbow to a wrist of the patient.

In some aspects, the techniques described herein relate to a blood pressure monitoring system, further including an attachment element configured to attach the exciter and the detector to a patient, wherein the exciter is positioned over a radial artery of the patient.

In some aspects, the techniques described herein relate to a blood pressure monitoring system, further including an attachment element configured to attach the exciter and the detector to a patient, wherein the detector is positioned over a radial artery of the patient.

In some aspects, the techniques described herein relate to a blood pressure monitoring system including: an exciter configured to produce an acoustic signal; a plurality of detectors spaced apart from the exciter, the plurality of detectors being configured to detect the acoustic signal and to produce a plurality of electrical output signals, wherein the plurality of detectors includes at least four detectors; and a processor configured to determine a blood pressure measurement from the plurality of electrical output signals.

In some aspects, the techniques described herein relate to a blood pressure monitoring system, wherein the plurality of detectors are separated by at least one gap.

In some aspects, the techniques described herein relate to a blood pressure monitoring system, wherein the plurality of detectors are mechanically decoupled from the exciter.

In some aspects, the techniques described herein relate to a blood pressure monitoring system, wherein the plurality of detectors are mechanically decoupled from one another.

In some aspects, the techniques described herein relate to a blood pressure monitoring system, wherein the plurality of detectors are arranged in a linear array.

In some aspects, the techniques described herein relate to a blood pressure monitoring system, wherein the plurality of detectors are arranged in a two-by-two array.

In some aspects, the techniques described herein relate to a blood pressure monitoring system, wherein, using the plurality of electrical output signals, the processor is further configured to determine an arterial propagation distance traveled by the acoustic signal and to compute the blood pressure measurement using the arterial propagation distance.

In some aspects, the techniques described herein relate to a blood pressure monitoring system, wherein a distance between an exciter and a detector corresponds to each of the plurality of detectors, and wherein the processor is further configured to determine whether the distance corresponds to the arterial propagation distance.

In some aspects, the techniques described herein relate to a blood pressure monitoring system, wherein the processor is further configured to determine the blood pressure measurement using the arterial propagation distance and a measured phase delay between an electrical input signal and one or more electrical output signals.

In some aspects, the techniques described herein relate to a blood pressure monitoring system, wherein one or more of the plurality of detectors is mechanically decoupled from the exciter and wherein one or more of the plurality of detectors is not mechanically decoupled from the exciter.

In some aspects, the techniques described herein relate to a blood pressure monitoring system, wherein the processor is further configured to use the one or more electrical output signals corresponding to the plurality of detectors which is not mechanically decoupled from the exciter to determine one or more characteristics of the acoustic signal.

In some aspects, the techniques described herein relate to a blood pressure monitoring system, wherein the processor is further configured to adjust the plurality of electrical output signals or the blood pressure measurement using the one or more characteristics of the acoustic signal.

In some aspects, the techniques described herein relate to a blood pressure monitoring system, wherein the blood pressure measurement includes systolic pressure, diastolic pressure, mean arterial pressure, or instantaneous arterial pressure.

In some aspects, the techniques described herein relate to a blood pressure monitoring system, wherein the electrical input signal includes a plurality of frequencies.

In some aspects, the techniques described herein relate to a blood pressure monitoring system, further including a memory device.

In some aspects, the techniques described herein relate to a blood pressure monitoring system, further including an inertial measurement unit (IMU).

In some aspects, the techniques described herein relate to a blood pressure monitoring system, further including an attachment element configured to attach the exciter and the plurality of detectors to a palm side of a wrist on a forearm of a patient.

In some aspects, the techniques described herein relate to a blood pressure monitoring system, further including an attachment element configured to attach the exciter and the plurality of detectors to a patient, wherein the exciter is closer to a hand of the patient than the plurality of detectors.

In some aspects, the techniques described herein relate to a blood pressure monitoring system, further including an attachment element configured to attach the exciter and the plurality of detectors to a patient, wherein the plurality of detectors are closer to a hand of the patient than the exciter.

In some aspects, the techniques described herein relate to a blood pressure monitoring system, further including an attachment element configured to attach the exciter and the plurality of detectors to a patient, wherein the exciter and the plurality of detectors are attached parallel to a length of an arm of the patient from an elbow to a wrist of the patient.

In some aspects, the techniques described herein relate to a blood pressure monitoring system, further including an attachment element configured to attach the exciter and the plurality of detectors to a patient, wherein the exciter is positioned over a radial artery of the patient.

In some aspects, the techniques described herein relate to a blood pressure monitoring system, further including an attachment element configured to attach the exciter and the plurality of detectors to a patient, wherein the plurality of detectors are positioned over a radial artery of the patient.

In some aspects, the techniques described herein relate to an exciter, including: a housing configured to provide structural support for the exciter, wherein the housing includes an opening; an exciter nob coupled to the housing and configured to adjust a position via the opening of the housing; a suspension spring coupled to the exciter nob and the housing, wherein the suspension spring extends and retracts in response to a driven force; an internal structure coupled to the suspension spring, wherein the internal structure is configured to provide structural support for the suspension spring; a movable component coupled to the suspension spring and the exciter nob, wherein the movable component is configured to adjust a position in response to the driven force, wherein the position of the movable component adjusts the position of the exciter nob; at least one magnet coupled to the internal structure, wherein the at least one magnet is configured to produce at least in part the driven force, wherein the driven force includes magnetic force; and a wire coil coupled to the movable component, wherein the wire coil is configured to receive an applied signal and generate an electric field in response to the applied signal, wherein the electric field interacts with the at least one magnet to drive the magnetic force.

In some aspects, the techniques described herein relate to an exciter, further including a signal receiver coupled to the suspension spring and the movable component, wherein the signal receiver is configured to obtain a secondary signal to replicate movement of the exciter nob.

In some aspects, the techniques described herein relate to a blood pressure monitoring system including: an exciter configured to produce an acoustic signal; a plurality of detectors spaced apart from the exciter, the plurality of detectors being configured to detect the acoustic signal and to produce a plurality of electrical output signals, wherein the plurality of detectors are mechanically or acoustically decoupled from one another; and a processor configured to determine a blood pressure measurement from the plurality of electrical output signals.

In some aspects, the techniques described herein relate to a blood pressure monitoring system including: an exciter configured to transmit a signal into tissue of a patient; and a detector configured to receive the signal that is reflected from an artery of the patient, wherein the blood pressure monitoring system is configured to detect changes in blood pressure.

In some aspects, the techniques described herein relate to a blood pressure monitoring system, used with any.

In some aspects, the techniques described herein relate to a blood pressure monitoring system, wherein the changes in blood pressure are detected according to a system response based on the transmitted signal and the received signal.

In some aspects, the techniques described herein relate to a blood pressure monitoring system, wherein the exciter is coupled to a first substrate and the detector is coupled to a second substrate.

In some aspects, the techniques described herein relate to a blood pressure monitoring system, wherein the first substrate and the second substrate are mechanically or acoustically decoupled.

In some aspects, the techniques described herein relate to a blood pressure monitoring system, wherein the decoupling occurs due to the first substrate being separated from the second substrate.

In some aspects, the techniques described herein relate to a blood pressure monitoring system, wherein the first substrate is separated from the second substrate with a gap that is parallel to an axis along the artery.

In some aspects, the techniques described herein relate to a blood pressure monitoring system, wherein the gap is parallel to the axis but off center from the axis.

In some aspects, the techniques described herein relate to a blood pressure monitoring system, wherein the first substrate includes a portion that is perpendicular to the axis.

In some aspects, the techniques described herein relate to a blood pressure monitoring system, wherein the detector includes a plurality of detectors.

In some aspects, the techniques described herein relate to a blood pressure monitoring system, wherein the plurality of detectors includes 2 or more detectors.

In some aspects, the techniques described herein relate to a blood pressure monitoring system, wherein the plurality of detectors includes 3 or more detectors.

In some aspects, the techniques described herein relate to a blood pressure monitoring system, wherein the plurality of detectors includes 4 or more detectors.

In some aspects, the techniques described herein relate to a blood pressure monitoring system, wherein each of the plurality of detectors is coupled to a substrate arm with a substrate portion having a series of bends.

In some aspects, the techniques described herein relate to a blood pressure monitoring system, wherein each of the series of bends is 90-degrees.

In some aspects, the techniques described herein relate to a blood pressure monitoring system, wherein the plurality of detectors are aligned with an axis along the artery.

In some aspects, the techniques described herein relate to a blood pressure monitoring system, wherein, in response to the blood pressure monitoring system being in a first configuration, the exciter is centered with the detector, and in response to the blood pressure monitoring system being in a second configuration, the exciter is off center with the detector.

In some aspects, the techniques described herein relate to a blood pressure monitoring system, wherein the exciter is at a first position along the tissue of the patient above the artery, and wherein the detector is at a second position along the tissue of the patient above the artery.

In some aspects, the techniques described herein relate to a blood pressure monitoring system, wherein the first position is farther away from a wrist of the patient than the second position.

In some aspects, the techniques described herein relate to a blood pressure monitoring system, wherein the signal is an acoustic signal.

In some aspects, the techniques described herein relate to a physiological monitor including a non-transitory data store storing data collected from at least one sensor and computer-executable instructions; a processor in communication with the at least one sensor and the non-transitory data store, wherein the computer-executable instructions, when executed by the processor, configure the processor to measure blood pressure and cardiac output, wherein the blood pressure and the cardiac output are measured by using a same device.

In some aspects, the techniques described herein relate to a blood pressure monitoring system, used with any.

In some aspects, the techniques described herein relate to a blood pressure monitoring system, wherein the computer-executable instructions further include instructions, when executed by the processor, configure the processor to measure the blood pressure from a system response of a body of the patient.

In some aspects, the techniques described herein relate to a blood pressure monitoring system, wherein the computer-executable instructions further include instructions, when executed by the processor, configure the processor to obtain the system response by causing transmission of a transmit signal into the body of the patient and receiving a received signal from the patient's body.

In some aspects, the techniques described herein relate to a blood pressure monitoring system, wherein the system response is associated with a change between the transmit signal and the receive signal.

In some aspects, the techniques described herein relate to a blood pressure monitoring system including a housing including: a sensor interconnect portion configured to receive a sensor connector; a processor, coupled to the sensor interconnect portion, and configured to compute at least a blood pressure measurement from physiological data; and a hub interconnect portion, coupled to the processor, and configured to receive a hub connector to provide the blood pressure measurement, wherein the housing is configured to: attach to a forearm of a patient; and connect to a sensor with the sensor interconnect portion.

In some aspects, the techniques described herein relate to a blood pressure monitoring system, wherein the housing further includes a disposable portion and a reusable portion, wherein the disposable portion includes an adhesive to attach to the forearm of the patient.

In some aspects, the techniques described herein relate to a blood pressure monitoring system including a sensor including a first disposable portion, and configured to attach to a forearm of a patient with the disposable portion; and a housing including: a reusable portion including: a processor, wherein the processor is coupled to the sensor; and a second disposable portion configured to attach to the forearm of the patient.

In some aspects, the techniques described herein relate to a blood pressure measurement system, wherein the sensor is further configured to obtain physiological measurements from a body of the patient.

In some aspects, the techniques described herein relate to a blood pressure monitoring system, wherein the processor is configured to cause transmission of a transmit signal by the sensor and receive a receive signal from the sensor, wherein the receive signal includes the physiological measurements.

In some aspects, the techniques described herein relate to a blood pressure monitoring system including: a substrate including: a first section; a second section, coupled to the first section, and including: a first portion; a second portion; a third portion; a fourth portion; and a fifth portion; a third section, coupled to the first section, and including: a first portion, a second portion, a third portion, and a fourth portion; and an exciter, coupled to the fourth portion of the third section, and configured to transmit a signal into tissue of a patient; and a reference detector, coupled to the third portion of the third section, and configured to receive the signal that is reflected from a body of the patient; and a detector, coupled to the third and fifth portions of the second section, and configured to receive the signal that is reflected from the body of the patient.

In some aspects, the techniques described herein relate to a blood pressure monitoring system, used with any.

In some aspects, the techniques described herein relate to a blood pressure monitoring system, wherein the first portion of the third section is perpendicular to the second portion of the third section, and is parallel to the third and fourth portions of the third section.

In some aspects, the techniques described herein relate to a blood pressure monitoring system, wherein the first portion of the second section is parallel to the third and fifth portions of the second section, and is perpendicular to the second and fourth portions of the second section.

In some aspects, the techniques described herein relate to a magnetic exciter, configured to push a nob.

In some aspects, the techniques described herein relate to a magnetic exciter, wherein the magnetic exciter operates as the exciter.

In some aspects, the techniques described herein relate to a magnetic exciter, further configured to push the nob according to a signal.

In some aspects, the techniques described herein relate to a magnetic exciter, further including a magnet

In some aspects, the techniques described herein relate to a magnetic exciter, wherein the magnet includes at least two magnets

In some aspects, the techniques described herein relate to a magnetic exciter, wherein a first of the magnets includes a magnetic orientation opposite of a second of the two magnets

In some aspects, the techniques described herein relate to a magnetic exciter, further including a spring

In some aspects, the techniques described herein relate to a magnetic exciter, wherein the spring includes at least one spring

In some aspects, the techniques described herein relate to a magnetic exciter, wherein the spring includes at least two springs

In some aspects, the techniques described herein relate to a magnetic exciter, wherein the spring includes a suspension spring

In some aspects, the techniques described herein relate to a magnetic exciter, wherein the spring includes a coiled spring

In some aspects, the techniques described herein relate to a magnetic exciter, further comprising a mass, coupled to the spring, and configured to apply force to ensure the nob presses into tissue of a patient.

In some aspects, the techniques described herein relate to a magnetic exciter, further including a bobbin, coupled to the nob and the spring, configured to adjust a position to push the nob.

In some aspects, the techniques described herein relate to a magnetic exciter, further including a wire coil, coupled to the bobbin, configured to generate an electric field to adjust a position of the magnet

In some aspects, the techniques described herein relate to a magnetic exciter, further configured to receive a signal that causes the wire coil to generate the electric field.

In some aspects, the techniques described herein relate to a magnetic exciter, further comprising a wire connect, coupled to the wire coil, configured to receive a signal to apply to the wire coil.

In some aspects, the techniques described herein relate to a plurality of detectors, wherein the plurality of detectors are configured to obtain a signal at different times, wherein a time delay of a received signal between each of the detectors provide a variable consistent between each of the detectors to process the signal and cause computing of a blood pressure measurement.

In some aspects, the techniques described herein relate to a plurality 116, wherein the plurality.

In some aspects, the techniques described herein relate to a plurality 116-117, wherein a difference between the time delays for each of the received signals at each of the detectors provides information about a system response in which the plurality of detectors are placed.

In some aspects, the techniques described herein relate to a plurality 116-118, wherein the time delay between each of the plurality of detectors vary according to a plurality of physiological characteristics, wherein the plurality of physiological characteristics include at least one of a patient's skin characteristics, body composition, arterial placement, arterial size, and physiological parameters.

In some aspects, the techniques described herein relate to a method including: transmitting a signal into a wearer from an emitter; receiving the signal with a detector; obtaining a system response corresponding to a relation of the signal as transmitted and received; computing a blood pressure of the wearer according to the system response; and providing the blood pressure to the wearer.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the disclosure.

FIG. 1 illustrates an example embodiment of a blood pressure monitoring system.

FIGS. 2A, 2B, 2C illustrate the sensor illustrated in FIG. 1 in various configurations, including in the as-worn position on a patient's forearm.

FIG. 3 illustrates a diagram representing an example operating mode for a sensor in accordance with aspects of the present disclosure.

FIGS. 4A-4B illustrate example embodiments of sensors for a blood pressure monitoring system in accordance with aspects of the present disclosure.

FIG. 4C illustrates a perspective view of the sensor of FIG. 4B in accordance with aspects of this disclosure.

FIG. 4D illustrates a top view of the sensor of FIG. 4B in accordance with aspects of this disclosure.

FIG. 4E illustrates a bottom view of the sensor of FIG. 4B in accordance with aspects of this disclosure.

FIG. 4F illustrates a side view of the sensor of FIG. 4B in accordance with aspects of this disclosure.

FIG. 4G illustrates a front view of the sensor of FIG. 4B in accordance with aspects of this disclosure.

FIGS. 5A-5B illustrate an example embodiment of an exciter for the sensors in accordance with aspects of the present disclosure.

FIGS. 6A-6B illustrate an example embodiment of an exciter for the sensors in accordance with aspects of the present disclosure.

FIGS. 7A-7B illustrate example embodiments of an exciter for the sensors in accordance with aspects of the present disclosure.

FIGS. 7C-7D illustrates a graph of magnetic characteristics for the exciter in FIG. 7A in accordance with aspects of the present disclosure.

FIGS. 8A, 8B, 8C illustrate example embodiments of an exciter for the sensors in accordance with aspects of the present disclosure.

FIG. 9 illustrates a block diagram for an example embodiment of a blood pressure monitoring system in accordance with aspects of the present disclosure.

FIGS. 10A-10B illustrate an example embodiment of a blood pressure monitoring system with a built-in display in accordance with aspects of the present disclosure.

FIGS. 11A-11C illustrate an example embodiment of a blood pressure monitoring system with an exciter and detectors integrated in a wristband in accordance with aspects of the present disclosure.

FIGS. 12A-12C illustrate an example embodiment of a blood pressure monitoring system which is similar to that of FIGS. 11A-11C, but which additionally includes a built-in display in accordance with aspects of the present disclosure.

FIG. 13 illustrates a flow chart depicting an example routine for operating a sensor in accordance with aspects of the present disclosure.

### DETAILED DESCRIPTION OF THE PREFERRED EMBODIMENT

Various embodiments of systems and methods for continuously and noninvasively measuring a patient's blood pressure are described herein.

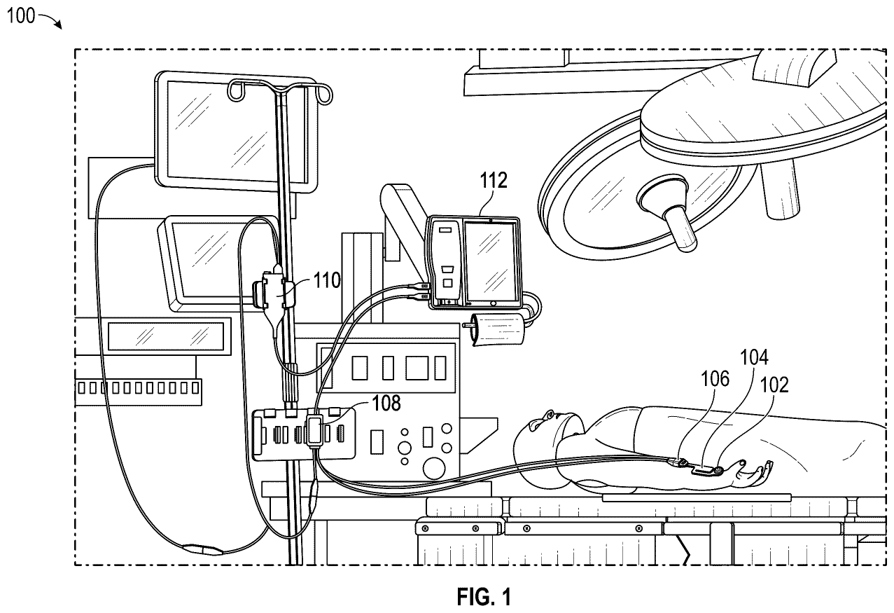

FIG. 1 illustrates an example embodiment of a blood pressure monitoring system 100 for a patient. The blood pressure monitoring system 100 can be used to noninvasively monitor the patient's blood pressure in an artery, as described further herein. The blood pressure monitoring system 100 can provide a relatively continuous (such as real-time) measurement waveform of the patient's blood pressure. In some embodiments, the blood pressure monitoring system 100 may provide measurements of the instantaneous blood pressure, the systolic blood pressure, the diastolic blood pressure, the mean arterial pressure, and/or any other blood pressure metric. In some embodiments, the measurement output of the blood pressure monitoring system 100 can be compared with that of an invasive direct arterial line, and can be contrasted with the more intermittent measurements provided by a noninvasive cuff-based measurement device.

As illustrated in FIG. 1, the blood pressure monitoring system 100 may include an exciter 102, a detector 104, an RD connector 106, a connectivity hub interface 108, a device interconnect 110, and a patient monitoring platform 112. In some cases, the exciter 102, the detector 104, and RD connector 106 may be positioned spaced apart from one another but adjacent to the radial artery in a patient's arm. The exciter 102 and the detector 104 may be coupled to the connectivity hub interface 108 using the RD connector 106. The connectivity hub interface 108 may be coupled to the device interconnect 110 and the patient monitoring platform 112. In some instances, the blood pressure monitoring system 100 may include other elements and topologies. For example, the blood pressure monitoring system 100 may include a signal/waveform generator connected to the exciter 102 to provide a desired input signal, as well as a processor connected to the detector 104 to analyze the detected output signal. In addition, the blood pressure monitoring system 100 can include a power source, such as a battery, a digital-to-analog converter (DAC) to convert a digital signal from the signal/waveform generator to an analog electrical signal, an analog-to-digital converter (ADC) to convert an electrical signal from the detector 104 to a digital signal, a display, signal leads, an attachment tape, strap, or other element for attaching the system to the patient. In some instances, the blood pressure monitoring system 100 may include an accelerometer, and/or gyroscope (such as, for example, an inertial measurement unit (IMU)). These devices can be used to determine whether the patient is moving, which may result in an inaccurate blood pressure measurement. For example, if the accelerometer and/or gyroscope may detect a degree of motion above a selected threshold, blood pressure data or measurements collected during the motion may be disregarded or deemphasized by the blood pressure monitoring system 100. In some examples, the blood pressure monitoring system 100 may have various topologies. For example, as illustrated in FIG. 1, a first topology may include the components as shown. In other topologies, the components may be combined and/or omitted. For example, the connectivity hub interface 108 may be in a same housing as the device interconnect 110. In other examples, the connectivity hub interface 108 and the device interconnect 110 may be part of the patient monitoring platform 112 (for example, housed within the same device).

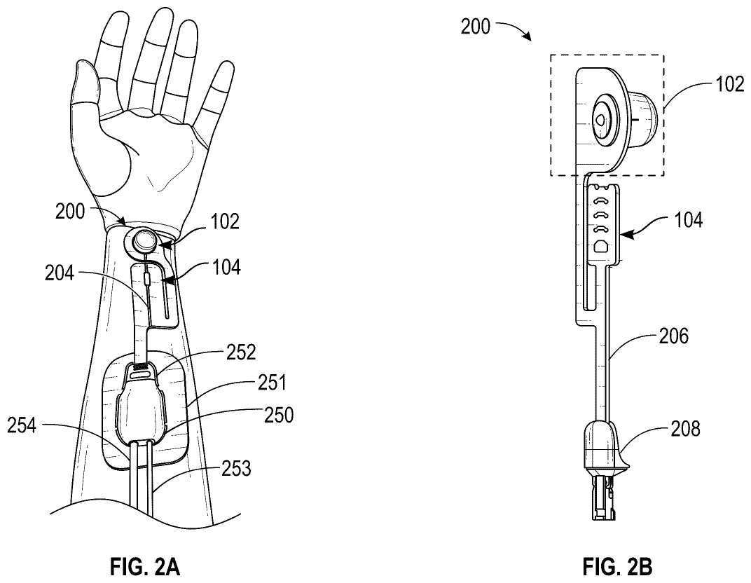

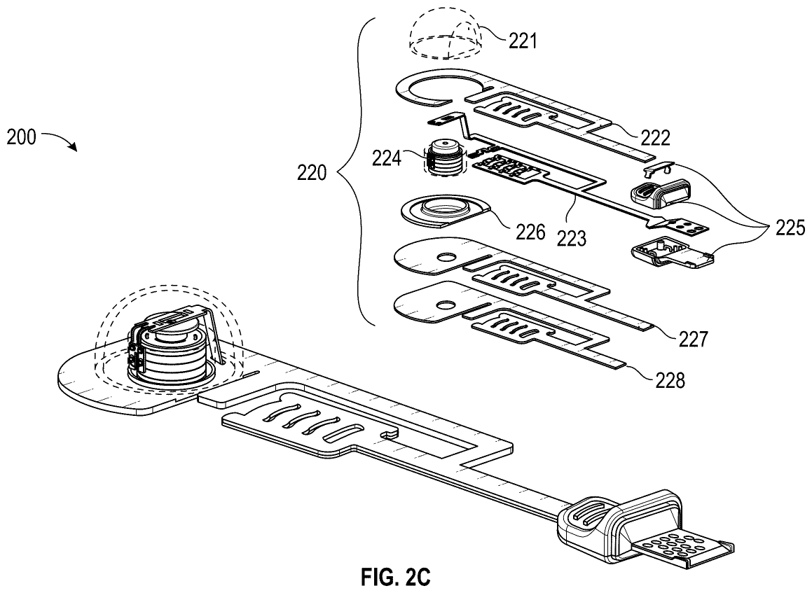

FIG. 2A illustrates an example embodiment of a blood pressure monitoring system 200 in an as-worn position on a patient's forearm. As shown, FIG. 2A illustrates the exciter 102, the detector 104, an alignment indicator 204, and a hub interconnect portion having a first portion 250 and a second portion 251, located on the inside of the patient's forearm. In some instances, the position of the components may be proximate to an artery of the patient, such as, for example the radial artery or the ulnar artery. For example, the exciter 102 and the detector 104 can be placed directly over the radial artery.

In the illustrated embodiment, the exciter 102 and the detector 104 may be attached to the arm with adhesive provided on the bottom surface of the blood pressure monitoring system 200. The substrate of the blood pressure monitoring system 200 can include portions which extend laterally away from the measurement axis of the sensor and wrap at least partially around the patient's arm to help fix the sensor securely in place.

The exciter 102 and the detector 104 may be separated from one another along a length of part of the patient's body. For example, the length may include a distance across the patient's body, such as along an arm of the patient (along the radial artery) by a separation distance. In some cases, the distance may be 0-5 cm, 5-10 cm, 10-15 cm, 15-20 cm, 20-30 cm, 0-10 cm, 0-15 cm, 0-20 cm, 0-25 cm, and/or 0-30 cm, though other separation distances are also possible.

In some instances, the exciter 102 and the detector 104 can be acoustically and/or mechanically coupled with the patient's body. In some examples, the exciter 102 can be a transducer which converts electrical energy from a power source (such as an electrical signal from an electrical signal/waveform generator) into acoustic and/or mechanical energy. The acoustic and/or mechanical energy may be an input acoustic signal, emitted into the patient. In some embodiments, the exciter 102 may be a piezo device or a microelectromechanical system (MEMS). Other types of exciters can also be used. The exciter 102 may emit the input acoustic signal, which is coupled into the body and then propagates via multiple paths to the detector 104. The detector 104 can be a transducer which converts acoustic and/or mechanical energy into electrical energy as an electrical output signal. In some embodiments, the detector 104 may be a piezo device or a MEMS device, though other types of acoustic detectors can be used. The electrical output signal from the detector 104 can be provided to a processor (for example, after being digitized) for analysis by one or more algorithms.

The hub interconnect portion may include a first portion 250 and a second portion 251. The first portion 250 may include a connection portion that couples to an RD connector of the sensor. For example, the first portion 250 may couple to the hub interface (such as, hub interface 108 in FIG. 1 as disclosed herein). In some examples, the first portion 250 may include a processor that processes sensor data from sensor 200. The first portion 250 may be reusable and detachable. For example, the first portion 250 may attach to the second portion 251 to hold the first portion 250 (for example, as a reusable monitor) in place. In this way, the second portion 251 is disposable and the first portion 250 is reusable. In some cases, the first portion 250 may include a connector portion 252 for the sensor 200 and/or cables 253, 254. The second portion 251 may be a disposable portion to adhere to a patient's body. For example, the second portion 251 may be prepackaged portion with an adhesive that may attach to a patient's body. In some cases, after the patient's blood pressure measurement is complete, the second portion 251 may be removable from the patient's body and discarded.

In some instances, the blood pressure monitoring system 200 may include the alignment indicator 204. The alignment indicator 204 can be used by a clinician to align the measurement axis of the blood pressure monitoring system 200 (i.e., the axis from the exciter 102 to the detector 104) with the patient's artery. In some embodiments, the clinician who is placing the blood pressure monitoring system 200 on the patient's arm can identify the location of the artery using, for example, palpation or ultrasound. In some cases, the clinician may mark the position of the artery with ink on the patient's arm. The clinician may then view the location of the artery and position the blood pressure monitoring system 200 such that the alignment indicator 204 lines up with the artery and additionally, the exciter 102 and the detector 104 are aligned. In this way, the clinician can ensure that the measurement axis of the blood pressure monitoring system 200 is aligned with the artery. This can improve signal-to-noise ratio and measurement accuracy.

Although FIG. 2A illustrates alignment of the blood pressure monitoring system 200 with the radial artery, the systems described herein can also be used with other arteries, measurement sites, and configuration. For example, the blood pressure monitoring system 200 may include an attachment element configured to attach the exciter 102 and the detector 104 to a palm side of a wrist on a forearm of a patient. In some cases, the blood pressure monitoring system 200 may attach the exciter 102 and the detector 104 to the patient with the exciter 102 being closer to a hand of the patient than the detector 104. In some cases, the blood pressure monitoring system 200 may attach the exciter 102 and the detector 104 to a patient, wherein the detector 104 is closer to a hand of the patient than the exciter 102. In some cases, the blood pressure monitoring system 200 may attach the exciter 102 and the detector 104 to a patient, wherein the exciter 102 and the detector 104 are attached parallel to a length of an arm of the patient from an elbow to a wrist of the patient. In some cases, the blood pressure monitoring system 200 may attach the exciter 102 and the detector 104 to a patient, wherein the exciter 102 is positioned over an artery of the patient. In some cases, the blood pressure monitoring system 200 may attach the exciter 102 and the detector 104 to a patient, wherein the detector 104 is positioned over the artery of the patient.

FIG. 2B illustrates another view of the blood pressure monitoring system 200. As shown, the blood pressure monitoring system 200 may include the exciter 102, detector 104, a substrate 206, and an interconnect portion 208. The substrate 206 may include multiple layers, including a flexible circuit layer. Top and bottom layers of the substrate may be formed of a flexible material, such as relatively thin layers of foam, silicone, plastic, or any other material as disclosed herein. The bottom layer may include adhesive on its bottom surface to enable the blood pressure monitoring system 200 to be attached to a patient's body. The adhesive may be covered by a peel off cover which can be removed by medical personnel when placing the sensor. The bottom layer can also include a window directly underneath the exciter, the detector 104 to allow them better access to the patient's skin. A flexible circuit can be provided between the top and bottom layers of the substrate. The flexible circuit can include electrical traces to electrically connect the exciter 102 and the detector 104 to the interconnect portion 208, which can in turn connect to the battery, processor, signal/waveform generator, etc. A foam pad can be provided under the interconnect portion 208 for strain relief. The interconnect portion 208 may include a sensor-to-cable connection with tactile and audible feedback to ensure proper connection. In some cases, the interconnection portion 208 may provide signals (and/or data) to and from the exciter 102 and detector 104.

FIG. 2C illustrates another view of the blood pressure monitoring system 200. The blood pressure monitoring system 200 may include components 220, including a cover 221, first layer 222, sensor 223, exciter 224, connector components 225, support component 226, second layer 227, and third layer 228. In some examples, the cover 221 may provide protection and structural support to the underlying components of the blood pressure monitoring system 200, for example, the exciter 224. The cover 221 may include materials such as silicone, plastic, or other bio-compatible substances that ensure both flexibility and resilience. The cover 221 may provide a barrier for parts of the sensor 223 and the exciter 224 against external environmental factors, such as physical contact and moisture. Additionally, the cover 221 may maintain a fit over the components, ensuring they remain securely in place during use of the blood pressure monitoring system 200. In this way, the cover 221 may maintain accuracy and reliability of the blood pressure measurements, as it minimizes the risk of displacement or misalignment of the sensor elements.

In some examples, the first layer 222, second layer 227, and/or the third layer 228 may provide structural integrity and facilitate the secure attachment of the system 200 to the patient's body. The first layer 222 may include a flexible, biocompatible material such as silicone, foam, or a similar polymer, which allows the first layer 222 to conform to the contours of the patient's skin, ensuring a snug fit and minimizing movement during use. The first layer 222 may couple to the sensor 223 and the support component 226. Additionally, the first layer 222 may incorporate openings or windows that align with the exciter and detector components, allowing these elements to maintain direct contact with the skin for optimal signal transmission and detection. In some cases, the second layer 227 and/or the third layer 228 may include an adhesive surface, which may be covered by a removable protective film that can be peeled away prior to application. In this way, the adhesive may be gentle on the skin while providing sufficient adhesion to maintain the position of the monitoring system throughout the duration of use.

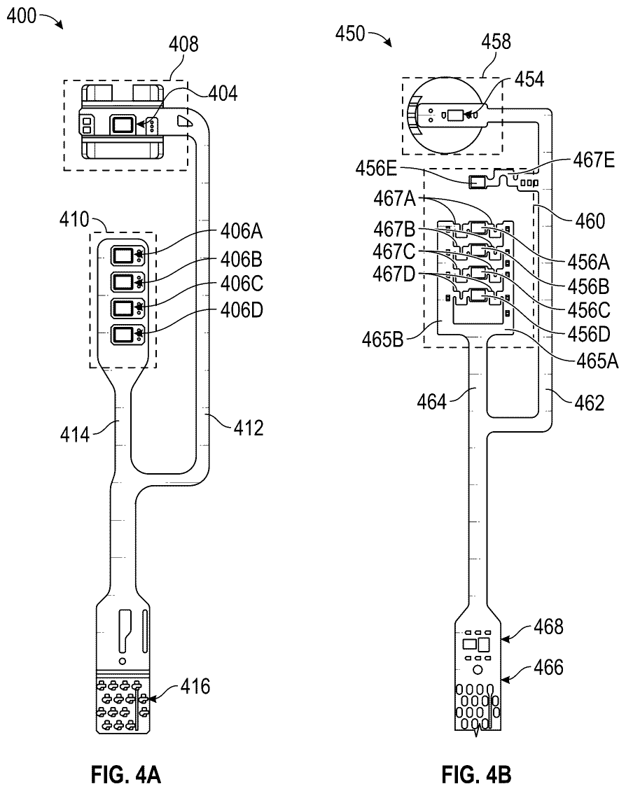

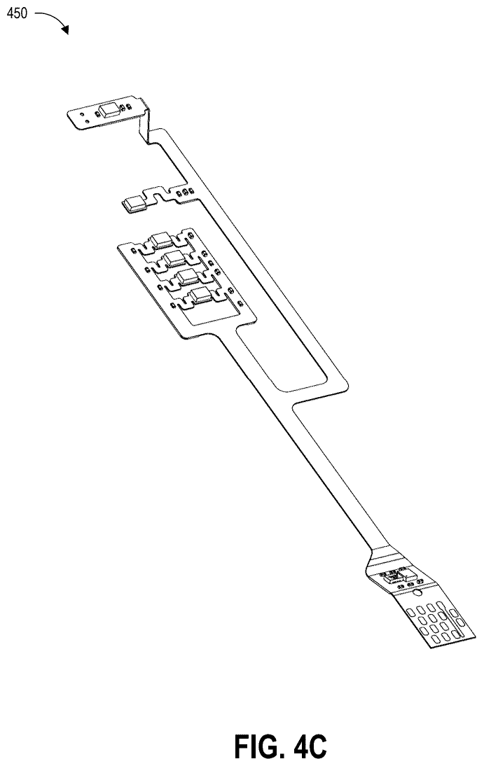

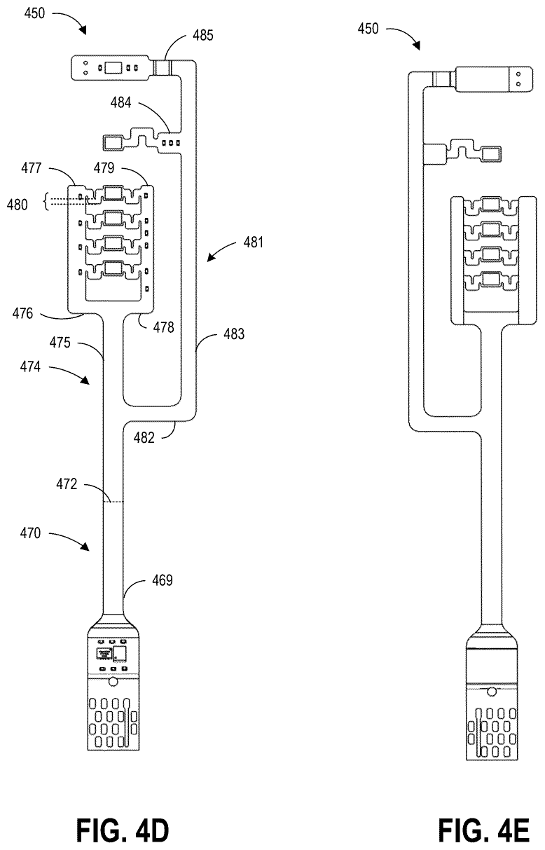

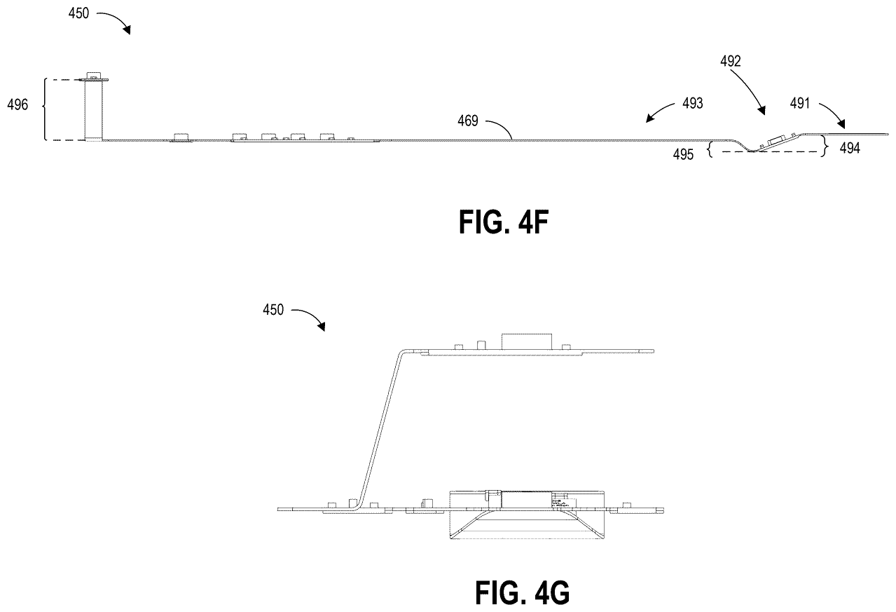

In some examples, the sensor 223 may noninvasively measure a patient's blood pressure by providing and detecting acoustic signals from the patient's body. The sensor 223 may be positioned to ensure contact with the patient's skin, allowing the sensor 223 (for example, via detectors as described herein) to capture the acoustic signals generated by the exciter 224. The sensor 223 may include materials, components, features, and/or functions, as described herein (for example, the sensor 400, 450 in FIGS. 4A and 4B).

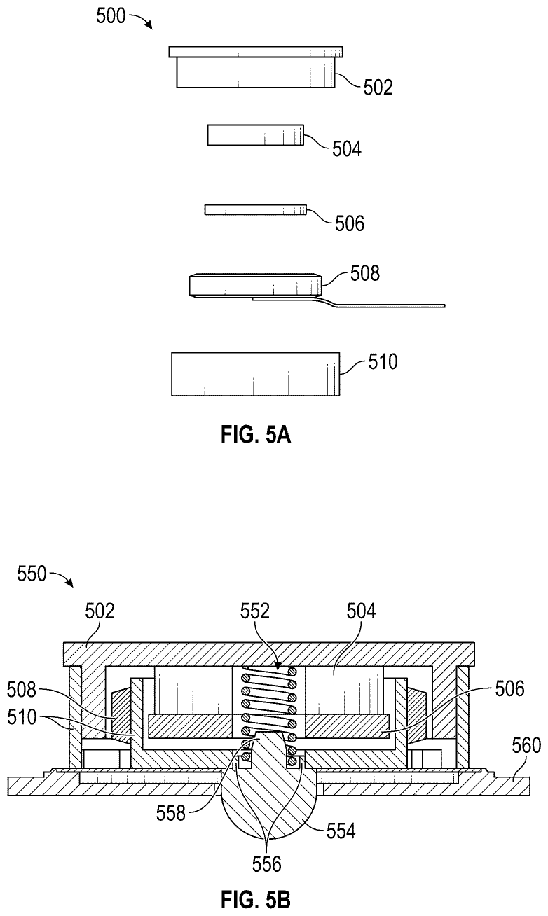

In some examples, the exciter 224 may generate a signal that propagates through the patient's body to facilitate noninvasive blood pressure measurement (for example, detection by the sensor 223). The exciter 224 may include materials, components, features, and/or functions, as described herein (for example, the exciter 500, 600, 700, 800 in FIGS. 5A-8C).

In some examples, the connector components 225 may provide an interface between the exciter 224, sensor 223, and the broader system architecture (such as, an interconnect hub 108 as disclosed herein). The connector components 225 may ensure reliable electrical connectivity, facilitating the transmission of signals from the exciter 224 to the sensor 223 to a processor for analysis. The connector components 225 may include a variety of connection types, such as snap-fit connectors, soldered joints, or flexible circuit connections, each chosen based on the specific application requirements and environmental conditions. The design of the connector components 225 may correspond to factors such as mechanical stability, electrical conductivity, and ease of assembly, ensuring that the connections remain secure and effective throughout the device's operational life.

In some examples, the support component 226 may provide structural stability and support for the exciter 224 within the blood pressure monitoring system 200. The support component 226 may ensure the exciter 224 remains positioned after placement of the system 200, reducing potential displacement that could affect the accuracy of the signal generation and detection. The support component 226 may include durable, biocompatible material such as a high-strength polymer or metal alloy, which is capable of withstanding the mechanical stresses encountered during the exciter's operation.

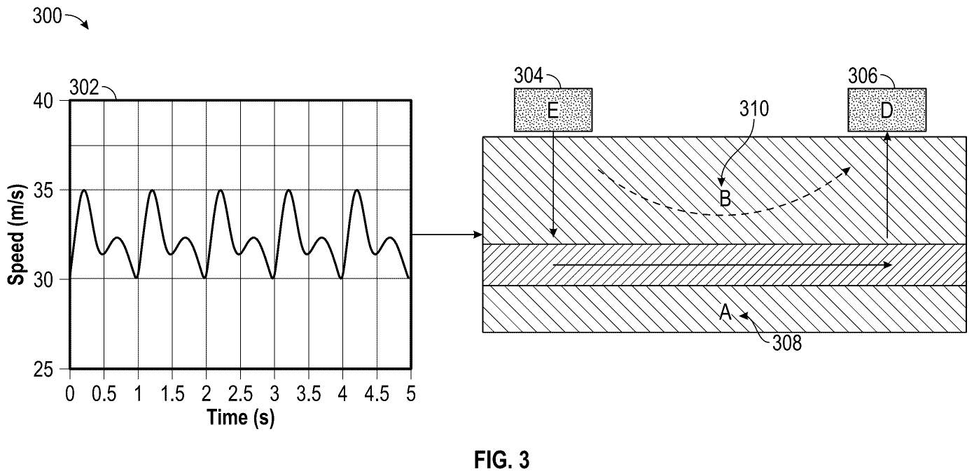

FIG. 3 illustrates a diagram 300 representing an example method of operation for a sensor. As shown, FIG. 3 schematically illustrates propagation of a signal from an exciter 304 (for example, exciter 102 in FIGS. 1-2C) through the patient's body to a detector 306 (for example, detector 104 in FIGS. 1-2C). The signal may include at least one of an acoustic signal, an electrical signal, a magnetic signal, an RF signal, and/or a mechanical signal, or another signal appropriate for measuring blood pressure. As shown in FIG. 3, the exciter 304 and the detector 306 can be placed in contact with the patient's skin over an artery (as illustrated with the arrow with path “A” 308). A distance separates the exciter 304 and the detector 306 (such as the separation distance as disclosed herein). The signal from the exciter 304 can propagate to the detector 306 via multiple different paths. For example, as shown in FIG. 3, a component of the signal can propagate from the exciter 304 to the detector 306 primarily via the artery, as shown with path “A” 308. Path “A” 308 may vary, for example, in depth, size, blood flow (such as speed and volume), etc., corresponding with the patient's body (as illustrated in graph 302, showing changes in blood speed over time). Other components of the signal may propagate to the detector 306 via bone, skin, muscle, and other tissues in the body, as shown with path “B” 310. Though not illustrated, still other components of the signal may propagate to the detector 306 via external structures, such as sensor substrates, adhesive tape, etc. which may be part of a blood pressure measurement system itself. Such signal paths may also be characterized as bypass paths.

Propagation of the emitted signal may be impacted by the various paths of the patient's body. The component of the output signal which propagates from the exciter 304 to the detector 306 primarily via the artery is affected by the instantaneous blood pressure within the artery. This is because the instantaneous blood pressure in the artery affects the stiffness of the arterial walls, which in turn affects the speed of the signal which propagates via the artery. Because the instantaneous blood pressure in the artery pulses with the patient's heartbeat, the signal which propagates via the artery is modulated by the patient's pulse.

The systems as described herein may determine a system response of how the signal received at the detector 306 may correspond to the emitted signal across the path “A” 308 and path “B” 310. In some instances, the signal received at the detector 306 may be represented as a relation of the emitted signal (“E”) and a combination of the component of the signal which propagates via the patient's body. In some examples, the blood pressure systems as described herein may rely on a relation between a signal transmitted by the exciter 306 and the signal received by the detector 304. For example, in some cases, the blood pressure systems may identify the relation between the signal transmitted to received as follows:

$${D}_{x} ≅ {E}_{o} {e}^{- j B ( t )}$$

where Dₓ is a signal received by the detector 304, Eₒ is the signal provided by the exciter 306, e^{-jB(t)} is interaction along the path 310. In other cases, the blood pressure systems may identify the relation between the signal transmitted to received as follows:

$$D = E * ( A ( t ) + B )$$

where D is a signal received by the detector 304, E is the signal provided by the exciter 306, A(t) is arterial blood flow according to time, and B is impact to the detected signal by the path 310 (for example, the interference of the signal from the patient's body).

In some examples, the blood pressure systems as described herein may compute a blood pressure from signals (for example, transmitted into the body of a patient and received from the patient). The blood pressure systems may compute an equation that may allow for determining the blood pressure of the patient, for example, by separating signal components of the detected signal (which may have been impacted by the various paths). The modulation introduced by the pulsing of the artery allows the component of the output signal which propagated to the detector 306 via the artery to be separated from the other components of the output signal which propagated to the detector 306 via other paths. In some embodiments, the processing algorithm(s) implemented by the processor which receives the output of the detector 306 may be used to isolate the component of the output signal which propagated primarily via the artery from the other components. In some embodiments, the modulation introduced by the pulsing of the artery can be understood as modulation which introduces a phase and/or time delay in the signal as it propagates from the exciter 304 to the detector 306.

FIGS. 4A-4B illustrate example embodiments of sensors for a blood pressure monitoring system. FIG. 4A illustrates an example embodiment of a sensor 400 (for example, used in the blood pressure monitoring system 100,200 of FIGS. 1-2). The sensor 400 may include a feedback detector 404, detectors 406A-D, an exciter portion 408, a detector portion 410, a first bypass portion 412, a second bypass portion 414, and an connector 416.

In some instances, the feedback detector 404 may detect a signal emitted from an exciter (such as exciters depicted in FIGS. 5A-8C). In some instances, the exciter may emit a signal into a patient's body when the sensor 400 is worn by the patient. The respective purpose of the feedback detector 404 adjacent to the exciter, and spaced apart from the detectors 406A-D, is to obtain received signals from the exciter, without interference from the patient's body. The signal may include at least one of an acoustic signal, an electrical signal, a magnetic signal, RF signal, and/or mechanical signal, or another signal appropriate for measuring blood pressure. The feedback detector 404 may detect the signal at a time before the signal is detected by the detectors 406A-D. In this way, the detected signal may be almost an exact received signal as the emitted signal. The presence of the feedback detector 404 may allow for signal processing that provides a basis for computing blood pressure of the patient.

In some instances, the feedback detector 404 may be a transducer, being able to convert the emitted signal from the exciter from the type of emitted signal to a type of received signal. For example, if the emitted signal is acoustic, the feedback detector 404 may convert the received acoustic signal into an electrical signal. In this manner, the feedback detector 404 be made of a piezoelectric material to convert the mechanical energy from the acoustic signal to electrical energy, to produce the electrical signal. In some examples, the signal may be an acoustic signal, electrical signal, magnetic signal (such as magnetic resonance), RF signal, or another signal appropriate for measuring blood pressure.

The feedback detector 404 may be located directly adjacent to the exciter. For example, the feedback detector 404 may be spaced apart from the exciter within a distance of 5 mm, within 2 mm, or within 1 mm. In some examples, the feedback detector 404 may be located to the right (or left, below, above) of the exciter and aligned with a horizontal (or vertical) axis located central to the exciter. In some examples, the sensor 400 may include more than one feedback detector. In some cases, when the sensor 400 includes more than one feedback detector, the position of the feedback detectors may be linearly or radially positioned (or another array placement method, such as a linear array) around the exciter.

In some cases, the detectors 406A-D may detect the signal emitted from the exciter (such as exciters depicted in FIGS. 5A-8C). The detectors 406A-D may allow for signal processing that provides a basis for computing blood pressure of the patient. In some instances, the exciter may emit a signal into a patient's body when the sensor 400 is worn by the patient. For example, the sensor 400 may be placed on a palm side of the patient's wrist, with the exciter within a distance of the patient's palm. In some cases, the exciter and the detectors 406A-D may be spaced parallel to the patient's arm to elbow side of the forearm. In the illustrated embodiment, the exciter is aligned with a central axis of the detectors 406A-D, though other layouts are also possible. For example, the sensor 400 may be placed on a palm side of the patient's wrist, with the exciter within a distance of the patient's palm and the detectors 406A-D aligned with an artery of the patient. In some cases, the exciter may be closer to the patient's wrist than the detectors 406A-D (and in some cases, the exciter may be farther away from the patient's wrist than the detectors 406A-D). The detectors 406A-D may detect the signal at a time after the signal is detected by the feedback detector 404. In this way, the detected signal is time delayed from a moment when the emitted signal was transmitted, and time delayed from a moment when the signal is received by the feedback detector 404.

The detectors 406A-D may be spaced apart such that the signal received by each of the individual detectors may be different from one another by a time delay (or phase delay), which provides the sensor 400 an ability to more precisely process the signal. The time delay of a received signal between each of the detectors may provide a variable consistent between each of the detectors to process the signal. The difference between the time for each received signal at each of the detectors may provide information about the system in which the detectors 406A-D are placed. For example, a known time delay between the detectors 406A-D may vary depending on characteristics of the patient's skin characteristics (for example, density, temperature, thickness), body composition (for example, muscle and fat mass), arterial placement, arterial size, etc.

In some cases, the detectors 406A-D may be arranged in an array positioned for receiving the emitted signal. In some examples, the array may consist of a linear array, grid array, non-linear array (such as triangular placement, circular placement, etc.), and/or the like. In some instances, the detectors 406A-D may be arranged in a two-by-two grid of four detectors. More or fewer detectors can also be used and the detectors 406A-D.

In some instances, the sensor 400 may include a substrate. In some cases, the exciter is provided on an exciter portion 408 of the substrate, while the detectors 406A-D are provided on a detector portion 410 of the substrate. In some cases, the substrate may include a flexible circuit. The substrate may include a first bypass portion 412 of the substrate and a second bypass portion 414 of the substrate. In some instances, the first and second bypass portions 412,414 of the substrate can be an elongate arm or lead type structures. The first and second bypass portions 412,414 may be connected to the exciter portion 408 and the detector portion 410 at their respective distal ends and are connected to one another at their proximal ends, near the connector 416 of the sensor. The first and second bypass portions 412,414 can be used to provide structural support for the exciter portion 408 and the detector portion 410 of the substrate. The first and second bypass portions 412,414 may do so in a manner that provides a bypass signal path from the exciter to the detectors 406A-D that is longer (or substantially longer) than the straight-line distance between the exciter and the detectors 406A-D.

In some instances, the substrate can include multiple layers, including the flexible circuit layer. Top and bottom layers of the substrate may be formed of a flexible material, such as relatively thin layers of foam, silicone, plastic, or any other material as disclosed herein. The bottom layer may include adhesive on its bottom surface to enable the sensor 400 to be attached to a patient's arm. The adhesive may be covered by a peel off cover which can be removed by medical personnel when placing the sensor. The bottom layer can also include a window directly underneath the exciter, the feedback detector 404, and the detectors 406 to allow them better access to the patient's skin. A flexible circuit can be provided between the top and bottom layers of the substrate. The flexible circuit can include electrical traces to electrically connect the exciter, the feedback detector 404, and the detectors 406A-D to the connector 416, which can in turn connect to the battery, processor, signal/waveform generator, etc. A foam pad can be provided under the connector 416 for strain relief.

The exciter may be provided on an exciter portion 408 of the substrate, while the detectors 406A-D is provided on a detector portion 410 of the substrate. The sensor 400 may include a first bypass portion 412 of the substrate and a second bypass portion 414 of the substrate. In some embodiments, the first and second bypass portions 412,414 of the substrate can be elongate arm or lead type structures. The first bypass portion 412 may include a distal end and a proximal end. The distal end may be connected to the exciter portion 408. The proximal end may be connected to the second bypass portion, near a connector end of the substrate, which in turn mechanically couples with the sensor's connector 416. This arrangement where the distal end of the first bypass portions 412 of the substrate is connected to the exciter portion 408 of the substrate, and the proximal ends connected to the second bypass portion 414 of the substrate, can be beneficial because the exciter can be more firmly held in a fixed spatial relationship with respect to the detectors 406A-D. The first and second bypass portions 412,414 can be used to provide structural support for the exciter portion 408 and the detector portion 410 of the substrate, but do so in a manner that provides a bypass acoustic signal path from the exciter to the detectors 406A-D that is longer (or substantially longer) than the straight-line distance between the exciter and the detectors 406A-D, as described in more detail below.

The exciter and the detectors 406A-D in sensor 400 may be separated to allow a signal emitted by the exciter to be detected by the feedback detector 404 and the detectors 406A-D. The distance between the exciter and the detectors 406A-D may be selected to provide more precise signal processing capability for the sensor 400. For example, the distance between the exciter and the detectors 406A-D may correspond with a frequency of signal emitted, such that the distance provides reduced noise. In some cases, higher frequencies can allow for the use of smaller separation distances. This in turn may allow for a smaller separation distance between the exciter and the detectors 406A-D. In some instances, the distance may be less than 50 cm, or other separation distances may also be used. In some cases, longer separation distances lengthen the measured propagation delay of an acoustic signal, which can improve signal-to-noise ratio. However, longer separation distances can also increase attenuation of the acoustic signal, which in turn can worsen the signal-to-noise ratio. This tradeoff of separation distance and signal frequency can be balanced according to each application of the blood pressure monitoring system.

In some instances, the exciter may be mounted on exciter portion 408 and the detectors 406A-D may be mounted on the detector portion 410, which are in turn connected to bypass portions (also may be referred to as “arms” or “leads”) 412,414 of the substrate, respectively. These substrate portions may include signal traces for conducting electrical input signals to the exciter and for conducting electrical output signals from the detectors 406A-D. In some embodiments, the substrate portions and signal traces may be flexible to allow the sensor 400 to conform to the patient's anatomy at a monitoring site.

To further improve performance, the first and second bypass portions 412,414 can include one or more acoustic materials and/or dampening masses designed to absorb vibrations and further acoustically isolate the exciter from the detectors 406A-D. Acoustic absorptive material and/or vibration dampening mass can be provided, for example, at any location along the loopback path from the exciter to the detectors 406A-D via any structure of the sensor.

In the illustrated embodiment, the first bypass substrate portion 412 of the substrate (connecting to the exciter) may be separated from the second bypass portion 414 of the substrate (connecting to the detectors 406A-D). The separation may be acoustic decoupling, physical separation, mechanically isolated, and/or electrically isolated. As illustrated, a gap (for example, in the direction from the exciter to the detectors 406A-D) between the two bypass substrate portions 412,414 such that they are mechanically and/or acoustically decoupled. In some cases, this may provide acoustic isolation between the exciter and the detectors 406A-D. In some instances, the gap between the first and second bypass substrate portions 412,414 extends toward the connector 416 of sensor 400 by a distance that is a multiple of a straight-line distance between the exciter and the detectors 406A-D. In some instances, the arrangement of the bypass arms/leads may provide attenuation of bypass acoustic signals which may otherwise propagate from the exciter to the detectors 406A-D via vibrations in the substrate portions of sensor 400. For example, the bypass arm/lead arrangement can be designed such that the path from the exciter to the detectors 406A-D via the patient's body (for example, in a straight line from exciter to detector) is shorter than the loopback path from the exciter to the detectors 406A-D via the substrate portions 412,414. In this case, due to the much greater path length, bypass signals which propagate from the exciter to the detectors 406A-D via the first and second bypass substrate portions 412,414 can be attenuated to a greater degree than the desired signal which travels to the detectors 406A-D via the artery. The length of the bypass portions may improve performance of the blood pressure monitoring system (such as the blood pressure monitoring system 100 of FIG. 1). In some embodiments, the sensor 400 can be designed such that the bypass path from the exciter to the detectors 406A-D via the first and second bypass substrate portions 412,414 is greater than 1×, greater than 2×, greater than 5×, or greater than 10× the physiological path distance (for example, the straight-line distance) between the exciter and the detectors 406A-D.

While FIG. 4A shows a physical gap between the first bypass substrate portion 412 for the exciter and the second bypass substrate portion 414 for the detectors 406A-D, in other embodiments it may be that a sensor for the blood pressure monitoring system may be designed with a shared substrate portion for both the exciter and the detectors 406A-D. For example, in such embodiments, there may instead be an acoustic barrier which can be achieved by providing an acoustic absorber material and/or dampening mass between the exciter and the detectors 406A-D. In still other embodiments, a combination of open space (also referred to as “gaps”), acoustical absorbing materials, and/or dampening masses can be used to provide mechanical decoupling and/or acoustic isolation between the exciter and the detectors 406A-D. In some instances, the isolation between the exciter and the detectors 406A-D may provide vibration piping, such that the signal received by each of the detectors 406A-D may be independent from (i.e., reduced noise from) the exciter and the other detectors.

In some instances, the sensor 450 may fit within a housing. The housing may include a protective cover can be provided over the exciter. The housing may include a layer of foam between the exciter and the protective cover to provide acoustic isolation and prevent resonant vibration of the protective cover.

FIG. 4B illustrates an example embodiment of a sensor 450 for the blood pressure monitoring system. FIG. 4B illustrates an example embodiment of a sensor 450 (for example, used in the blood pressure monitoring system 100,200 of FIGS. 1-2C). The sensor 450 may include a feedback detector 454, detectors 456A-E, an emitter region 458, a detector region 460, a first bypass substrate portion 462, a second bypass substrate portion 464, a first path 465A, a second path 465B, a connector 466, detector attachments 467A-E, and a memory and IMU component 468.

In some instances, the feedback detector 454 may detect a signal emitted from an exciter (such as exciters depicted in FIGS. 5A-8C). In some instances, the exciter may emit a signal into a patient's body when the sensor 450 is worn by the patient. The respective purpose of the feedback detector 454 adjacent to the exciter, and spaced apart from the detectors 456A-E, is to obtain received signals from the exciter, without interference from the patient's body. The signal may include at least one of an acoustic signal, an electrical signal, a magnetic signal, RF signal, and/or mechanical signal, or another signal appropriate for measuring blood pressure. The feedback detector 454 may detect the signal at a time before the signal is detected by the detectors 456A-E. In this way, the detected signal may be almost an exact received signal as the emitted signal. The presence of the feedback detector 454 may allow for signal processing that provides a basis for computing blood pressure of the patient. The feedback detector 454 may be similar (or the same) as the feedback detector 404 as described herein. In some examples, the feedback detector 454 may be aligned along an axis corresponding to an artery of the patient of the sensor 450. For example, the patient may have the sensor 450 attached to their forearm, such that the feedback detector 454 is adjacent to an artery and aligned along the axis associated with the artery. In some instances, the feedback detector 454 may be a transducer, being able to convert the emitted signal from the exciter from the type of emitted signal to a type of received signal. For example, if the emitted signal is acoustic, then the feedback detector 454 may convert the received acoustic signal into an electrical signal. In this manner, the feedback detector 454 be made of a piezoelectric material to convert the mechanical energy from the acoustic signal to electrical energy, to produce the electrical signal. In some examples, the signal may be an acoustic signal, electrical signal, magnetic signal (such as magnetic resonance), RF signal, or another signal appropriate for measuring blood pressure. The feedback detector 454 may be located directly adjacent to the exciter. For example, the feedback detector 454 may be spaced apart from the exciter within a distance of 5 mm, within 2 mm, or within 1 mm. In some examples, the feedback detector 454 may be located to the right (or left, below, above) of the exciter and aligned with a horizontal (or vertical) axis centered on to the exciter. In some examples, the feedback detector 454 may be off center from an axis of the detectors 456A-E. In some examples, the sensor 450 may include more than one feedback detector. In some cases, when the sensor 450 includes more than one feedback detector, the position of the feedback detectors may be linearly or radially positioned (or another array placement method, such as a linear array) around the exciter.

In some cases, the detectors 456A-E may detect the signal emitted from the exciter (such as exciters depicted in FIGS. 5A-8C). The detectors 456A-E may allow for signal processing that provides a basis for computing blood pressure of the patient. In some instances, the exciter may emit a signal into a patient's body when the sensor 450 is worn by the patient. For example, the sensor 450 may be placed on a palm side of the patient's wrist, with the exciter within a distance of the patient's palm. In some cases, the exciter and the detectors 456A-E may be spaced parallel to the patient's arm to elbow side of the forearm. The exciter may be aligned with an axis. for example, the axis may correspond to a central axis of the detectors 456A-E, an axis of an artery of the patient wearing the sensor 450, or another layout having an axis. For example, the sensor 400 may be placed on a palm side of the patient's wrist, with the exciter within a distance of the patient's palm and the detectors 456A-E aligned with an artery of the patient (such as, aligned with an axis of the artery). In some cases, the exciter may be closer to the patient's wrist than the detectors 456A-E (and in some cases, the exciter may be farther away from the patient's wrist than the detectors 456A-E). The detectors 456A-E may detect the signal at a time after the signal is detected by the feedback detector 454. In this way, the detected signal is time delayed from a moment when the emitted signal was transmitted, and time delayed from a moment when the signal is received by the feedback detector 454. The detectors 456A-E may be spaced apart such that the signal received by each of the individual detectors may be different from one another by a time delay (and/or frequency delay, and/or phase delay), which provides the sensor 450 an ability to more precisely process the signal. In some cases, the detectors 456A-E may be mechanically, acoustically, electrically, magnetically, physically, independent from each other. In some instances, each of the detectors 456A-E may be attached to a mechanically (and/or acoustically, electrically, magnetically, physically) decoupled flex circuit line so that each of the detectors 456A-E are independent of each other. The mechanical and acoustic decoupling occurs through use of a series of bends in the flex circuits 467A-E. The bends can be a series of bends (for example, 90-degrees) that reduce the mechanical/acoustic coupling along the flex circuit. In some examples, the detectors 456A-E may have no straight line access between a left side and a right side of the detector attachments 467A-E. In some examples, some (or all) of the detectors 456A-E may be attached to the second bypass portion 464. The second bypass portion 464 may include a branched flex circuit, including the first path 465A and the second path 465B. In some cases, some (or all) of the detectors 456A-E may be located between the first path 465A and the second path 465B of the flex circuit. The first path 465A and the second path 465B of the flex circuit may attach to some (or all) of the detectors 456A-E. The first path 465A and the second path 465B of the flex circuit may attach to some (or all) of the detectors 456A-E with the detector attachments 467A-E to each of the detectors 456A-E. The detectors 456A-E may be separated by a gap from each of the other detectors. In some cases, the gap between each of the detectors 456A-E (and between each of the detector attachments 467A-E) may include a material for providing additional decoupling (for example, foam- or fiber-based acoustic absorbers, tape adhesive with acoustic and/or vibration damping characteristics, electrically insulative materials, and/or an air-gap, and/or any other materials as disclosed herein). In some cases, some of the detectors 456A-E may be in an arrangement along the second bypass substrate portion 464 and the rest of the detectors 456A-E may be separated from the arrangement and coupled to the first bypass substrate portion 462. The detectors 456A-E may each be separated by a distance. for example, the distance between a first detector and a second detector may be the same or different than the distance between the second detector and a third detector. In some cases, at least some of the detectors 456A-E may have a same distance apart from the other detectors (for example, equal distances between at least some of the detectors). The distances between each of the detectors 456A-E may be between about 0 mm and about 50 mm, for example, between about 5 mm and about 45 mm, between about 10 mm and about 40 mm, between about 15 mm inch and about 35 mm, between about 20 mm and about 30 mm, between about 25 mm and about 25 mm, or any value or range between any of these values or ranges or any value or range bounded by any combination of these values, although values or ranges outside these values or ranges can be used in some cases. The time delay of a received signal between each of the detectors may provide a variable consistent between each of the detectors to process the signal. The difference between the time for each received signal at each of the detectors may provide information about the system in which the detectors 456A-E are placed. For example, a known time delay between each of the detectors 456A-E may vary depending on characteristics of the patient's skin characteristics (for example, density, temperature, thickness), body composition (for example, muscle and fat mass), arterial placement, arterial size, physiological parameters (for example, blood pressure), etc. In some cases, the detectors 456A-E may be arranged in an array positioned for receiving the emitted signal. In some examples, the array may consist of a linear array, grid array, non-linear array (for example, triangular placement, circular placement, etc.). In some instances, the detectors 456A-E may be arranged in a two-by-two grid of four detectors. More or fewer detectors can also be used and the detectors 456A-E may be arranged in different arrays, including linear arrays. In some cases, the detectors 456A-E may include five detectors. In this manner, the detectors 456A-E may include four detectors in a linear arrangement along the second bypass substrate portion 464 and a fifth detector separated from the linear arrangement and coupled to the first bypass substrate portion 462. In some cases, some of the detectors 456A-E may be attached to the second bypass portion 464 and the rest of the detectors 456A-E may be attached to the first bypass portion 462.

In some instances, the sensor 450 may include a substrate. In some cases, the exciter is provided on an emitter region 458 of the substrate, while the detectors 456A-E is provided on a detector region 460 of the substrate. In some cases, the substrate may include a flexible circuit. The substrate may include a first bypass portion 462 of the substrate and a second bypass portion 464 of the substrate. In some embodiments, the first and second bypass portions 462,464 of the substrate can be an elongate arm or lead type structures. The first and second bypass portions 462,464 may be connected to the emitter region 458 and the detector region 460 at their respective distal ends and are connected to one another at their proximal ends, near the connector 466 of the sensor. The first and second bypass portions 462,464 can be used to provide structural support for the emitter region 458 and the detector region 460 of the substrate. The first and second bypass portions 462,464 may do so in a manner that provides a bypass signal path from the exciter to the detectors 456A-E that is longer (or substantially longer) than the straight-line distance between the exciter and the detectors 456A-E. In some instances, the substrate can include multiple layers, including the flexible circuit layer. Top and bottom layers of the substrate may be formed of a flexible material, such as relatively thin layers of foam, silicone, plastic, etc. The bottom layer may include adhesive on its bottom surface to enable sensor 450 to be attached to a patient's arm. The adhesive may be covered by a peel off cover which can be removed by a clinician when placing the sensor. The bottom layer can also include windows directly underneath the exciter, the feedback detector 454, and the detectors 456A-E to allow them better access to the patient's skin, though this is not necessarily required. A flexible circuit can be provided between the top and bottom layers of the substrate. The flexible circuit can include electrical traces to electrically connect the exciter, the feedback detector 454, and the detectors 456A-E to the connector 466, which can in turn connect to the battery, processor, signal/waveform generator, etc. A foam pad can be provided under the connector 466 for strain relief. The exciter may be provided on an emitter region 458 of the substrate, while the detectors 456A-E is provided on a detector region 460 of the substrate. The sensor 450 may include a first bypass portion 462 of the substrate and a second bypass portion 464 of the substrate. In some embodiments, the first and second bypass portions 462,464 of the substrate can be elongate arm or lead type structures. The first bypass portion 462 may include a distal end and a proximal end. The distal end may be connected to the emitter region 458. The proximal end may be connected to the second bypass portion, near a connector end of the substrate, which in turn mechanically couples with the sensor's connector 466. This arrangement where the distal end of the first bypass portions 462 of the substrate is connected to the emitter region 458 of the substrate, and the proximal ends connected to the second bypass portion 464 of the substrate, can be beneficial because the exciter can be more firmly held in a fixed spatial relationship with respect to the detectors 456A-E. The first and second bypass portions 462,464 can be used to provide structural support for the emitter region 458 and the detector region 460 of the substrate, but do so in a manner that provides a bypass acoustic signal path from the exciter to the detectors 456A-E that is longer (or substantially longer) than the straight-line distance between the exciter and the detectors 456A-E.

The exciter and the detectors 456A-E in sensor 450 may be separated to allow a signal emitted by the exciter to be detected by the feedback detector 454 and the detectors 456A-E. The distance between the exciter and the detectors 456A-E may be selected to provide more precise signal processing capability for the sensor 450. For example, the distance between the exciter and the detectors 456A-E may correspond with a frequency of signal emitted, such that the distance provides reduced noise. The separation distance can also be dependent upon the acoustic signal frequency. For example, higher frequencies can allow for the use of smaller separation distances. This in turn may allow for a smaller distance between the exciter and the detectors 456A-E. In some instances, the distance may be less than 10 cm. Other separation distances may also be used. In some cases, longer separation distances lengthen the measured propagation delay of an acoustic signal, which can improve signal-to-noise ratio. However, longer separation distances can also increase attenuation of the acoustic signal, which in turn can worsen the signal-to-noise ratio. This tradeoff of separation distance and signal frequency can be balanced according to each application of the blood pressure monitoring system. In some examples, the frequency emitted by the exciter (and thereby detected by the detectors), may be associated with a carrier frequency. For example, in some cases, the frequency may be a resonant frequency of the carrier frequency. in some cases, the carrier frequency may be 93.75 Hz, with the resonant frequency being integer values multiplied by the carrier frequency. For example, the resonant frequency may be between about 1-100 times the carrier frequency.

In some instances, the exciter may be mounted on emitter region 458 and the detectors 456A-E may be mounted on the detector region 460, which are in turn connected to bypass portions (also referred to as “arms” or “leads”) 462,464 of the substrate, respectively. These substrate portions may include signal traces for conducting electrical input signals to the exciter and for conducting electrical output signals from the detectors 456A-E. In some embodiments, the substrate portions and signal traces may be flexible to allow the sensor 450 to conform to the patient's anatomy at a monitoring site. To further improve performance, the portions 462,464 can include one or more acoustic materials and/or dampening masses designed to absorb vibrations and further acoustically isolate the exciter from the detectors 456A-E. Acoustic absorbing material and/or vibration dampening mass can be provided, for example, at any location along the loopback path from the exciter to the detectors 456A-E via any structure of the sensor. In the illustrated embodiment, the first bypass portion 462 of the substrate (connecting to the exciter) may be separated from the second bypass portion 464 of the substrate (connecting to the detectors 456A-E). The separation may be acoustic decoupling, physical separation, mechanically isolated, and/or electrically isolated. As illustrated, a gap (such as in the direction from the exciter to the detectors 456A-E) between the two bypass substrate portions 462,464 such that they are mechanically and/or acoustically decoupled. In some cases, this may provide acoustic isolation between the exciter and the detectors 456A-E. In some instances, the gap between the first and second bypass substrate portions 462,464 extends toward the connector 466 of sensor 450 by a distance that is a multiple of a straight-line distance between the exciter and the detectors 456A-E. In some instances, the arrangement of the bypass arms/leads may provide attenuation of bypass acoustic signals which may otherwise propagate from the exciter to the detectors 456A-E via vibrations in the substrate portions of sensor 450. For example, the bypass arm/lead arrangement can be designed such that the path from the exciter to the detectors 456A-E via the patient's body (for example, in a straight line from exciter to detector) is shorter than the loopback path from the exciter to the detectors 456A-E via the substrate portions 462,464. In this case, due to the much greater path length, bypass signals which propagate from the exciter to the detectors 456A-E via the bypass substrate portions 462,464 can be attenuated to a greater degree than the desired signal which travels to the detectors 456A-E via the artery. The length of the bypass portions may improve performance of the blood pressure monitoring system 100. In some embodiments, sensor 450 can be designed such that the bypass path from the exciter to the detectors 456A-E via the substrate portions 462,464 is greater than 2×, greater than 5×, or greater than 10× the physiological path distance (for example, the straight-line distance) between the exciter and the detectors 456A-E.

The memory and IMU component 468 can include one or more memory devices and/or an IMU. The one or more memory devices may store data and/or instructions (such as computer-executable instructions). The memory device may include a dynamic and/or static random-access memory (RAM), programmable read-only memory (PROM), erasable programmable read-only memory (EPROM), electrically erasable programmable read-only memory (EEPROM), and the like. In some cases, the stored data can be processed and/or unprocessed physiological data or other types of data (for example, motion and/or location data) obtained from the IMU. In some implementations, the memory and IMU component 468 can store information indicative and/or related to one or more users. For example, in some implementations, the IMU may detect an orientation of the user's arm (when the sensor 450 is attached to the user's arm), such that when the measured orientation is above a predetermined angle, the sensor 450 alerts another device. The information regarding the orientation position, predetermined angle, and alert information may be stored in the memory and IMU component 468. The memory and IMU component 468 can include at least one IMU. The IMU may measure motion, orientation, and/or location of a user. In some examples, the IMU may determine motion, orientation, position and/or location of a user. Further, a processor may be configured to receive motion, orientation, position, and/or location data of a user from the at least one IMU. Additionally, the processor may determine motion, orientation, position, and/or location of a user based on data received from the at least one IMU. For example, the memory and IMU component 468 can include an IMU that can measure static and/or dynamic acceleration forces and/or angular velocity. By measuring static and/or dynamic acceleration forces and/or angular velocity, an IMU can be used to calculate movement and/or relative position of the sensor 450. The IMU can include one or more, and/or a combination of, for example, an AC-response accelerometer (for example, a charge mode piezoelectric accelerometer and/or a voltage mode piezoelectric accelerometer), a DC-response accelerometer (for example, capacitive accelerometer, piezoresistive accelerometer), a microelectromechanical system (MEMS) gyroscope, a hemispherical resonator gyroscope (HRG), vibrating structure gyroscope (VSG), a dynamically tuned gyroscope (DTG), fiber optic gyroscope (FOG), a ring laser gyroscope (RLG), and the like. An IMU can measure acceleration forces and/or angular velocity forces in one-dimension, two-dimensions, or three-dimensions. With calculated position and movement data, users of the sensor 450 and/or others (for example, medical personnel) may be able to map the positions or movement vectors of the sensor 450. Any number of IMU's can be used to collect sufficient data to determine position and/or movement of the sensor 450. Further, the memory and IMU component 468 can be configured to determine and/or keep track of steps and/or distance traveled by a user based on data from at least one IMU.

In some instances, the sensor 450 may fit within a housing. The housing may include a protective cover can be provided over the exciter. The housing may include a layer of foam between the exciter and the protective cover to provide acoustic isolation and prevent resonant vibration of the protective cover.

While FIG. 4B shows a physical gap between the first bypass substrate portion 462 for the exciter and the second bypass substrate portion 464 for the detectors 456A-E, in other embodiments it may be that a sensor for the blood pressure monitoring system 100 may be designed with a shared substrate portion for both the exciter and the detectors 456A-E. For example, in such embodiments, there may instead be an acoustic barrier which can be achieved by providing an acoustic absorber material and/or dampening mass between the exciter and the detectors 456A-E. In still other embodiments, a combination of open space (or “gaps”), acoustical absorbing materials, and/or dampening masses can be used to provide mechanical decoupling and/or acoustic isolation between the exciter and the detectors 456A-E. In some instances, the isolation between the exciter and the detectors 456A-E may provide vibration piping, such that the signal received by each of the detectors 456A-E may be independent from (i.e., reduced noise from) the exciter and the other detectors 456A-E.

FIG. 4C illustrates a perspective view of sensor 450. FIGS. 4D-4E illustrate top and bottom views of sensor 450. As shown, the sensor 450 may include a substrate 469 having a first section 470, a second section 474, and a third section 480. The substrate 469 may have a thickness between about 0 mm and about 50 mm, for example, between about 5 mm and about 45 mm, between about 10 mm and about 40 mm, between about 15 mm inch and about 35 mm, between about 20 mm and about 30 mm, between about 25 mm and about 25 mm, or any value or range between any of these values or ranges or any value or range bounded by any combination of these values, although values or ranges outside these values or ranges can be used in some cases.

The first section 470 may include a width 472 and a length. The width of the first section 470 may be between about 0 mm and about 50 mm, for example, between about 5 mm and about 45 mm, between about 10 mm and about 40 mm, between about 15 mm inch and about 35 mm, between about 20 mm and about 30 mm, between about 25 mm and about 25 mm, or any value or range between any of these values or ranges or any value or range bounded by any combination of these values, although values or ranges outside these values or ranges can be used in some cases. Each portion of the first section 470, second section 474, and third section 480 may have a width according to range of widths described for the width 472. The length of the first section 470 may be between about 0 cm and about 50 cm, for example, between about 5 cm and about 45 cm, between about 10 cm and about 40 cm, between about 15 mm inch and about 35 cm, between about 20 mm and about 30 mm, between about 25 cm and about 25 cm, or any value or range between any of these values or ranges or any value or range bounded by any combination of these values, although values or ranges outside these values or ranges can be used in some cases. Each portion of the second section 474 and third section 480 may have a length according to the range of lengths described for the first section 470.

The second section 474 may include a first portion 475, a second portion 476, a third portion 477, a fourth portion 478, and a fifth portion 479. Each of the first portion 475, the second portion 476, the third portion 477, the fourth portion 478, and the fifth portion 479 may have a width according to the range of widths as described herein (for example, according to the range of widths for the width 472). Additionally, each of the first portion 475, the second portion 476, the third portion 477, the fourth portion 478, and the fifth portion 479 may have a length according to the range of lengths as described herein (for example, as described for the range of lengths for the first section 470).

Each of the detectors 456A-D may attach to the third portion 477 and the fifth portion 479, for example, with the detector attachments 467A-D. The detector attachments 467A-D may have a width 480. The width 480 may be between about 0 mm and about 50 mm, for example, between about 5 mm and about 45 mm, between about 10 mm and about 40 mm, between about 15 mm inch and about 35 mm, between about 20 mm and about 30 mm, between about 25 mm and about 25 mm, or any value or range between any of these values or ranges or any value or range bounded by any combination of these values, although values or ranges outside these values or ranges can be used in some cases. In some cases, the detector attachment 467E may have a width according to the range of widths described for the width 480.

The detector attachments 467A-E may have one or more bends (from the series of bends). in some examples, the detector attachments 467A-E may have one bend, two bends, three bends, four bends, five bends, six bends, seven bends, eight bends, nine bends, ten bends, or any number of bends to connect the detectors 456A-E with the substrate 469 (for example, attaching each of the detectors 456A-E to the third portion 477 and the fifth portion 479).

The third section 481 may include a first portion 482, second portion 483, third portion 484, and fourth portion 485. Each of the first portion 482, second portion 483, third portion 484, and fourth portion 485 may have a width according to the range of widths as described herein (for example, according to the range of widths for the width 472). Additionally, each of the first portion 482, second portion 483, third portion 484, and fourth portion 485 may have a length according to the range of lengths as described herein (for example, as described for the range of lengths for the first section 470).

FIGS. 4F-4G illustrate a side view of the sensor 450 and a front view of the sensor 450, respectively. The substrate 469 may include a connector portion 491, an IMU and memory portion 492, and a sensor portion 493. In some cases, the substrate 469 may be flat or have various heights. For example, the connector portion 491 may be at a different height than the IMU and memory portion 492, and a different height than the sensor portion 493. The IMU and memory portion may be sloped downward from the connector portion 491 and then sloped upward to the sensor portion 493. In some cases, a length of the slope downward may be different than a length of the slope upward. The length of the slope downward may be between about 0 mm and about 50 mm, for example, between about 5 mm and about 45 mm, between about 10 mm and about 40 mm, between about 15 mm inch and about 35 mm, between about 20 mm and about 30 mm, between about 25 mm and about 25 mm, or any value or range between any of these values or ranges or any value or range bounded by any combination of these values, although values or ranges outside these values or ranges can be used in some cases. The length of the upward slope may be according to the range of lengths described for the downward slope for the IMU and memory portion 492. Moreover, a first angle of which the IMU and memory portion 492 is offset from the connector portion 491 may be smaller than a second angle of which the IMU and memory portion 492 is offset from the sensor portion. The first angle may be between about 0-degrees and 180-degrees, for example, between about 10-degrees and about 170-degrees, about 20-degrees and about 160-degrees, between about 30-degrees and about 150-degrees, about 40-degrees and about 140-degrees, between about 50-degrees and about 130-degrees, about 60-degrees and about 120-degrees, between about 70-degrees and about 110-degrees, about 80-degrees and about 100-degrees, between about 90-degrees and about 90-degrees, or any value or range between any of these values or ranges or any value or range bounded by any combination of these values, although values or ranges outside these values or ranges can be used in some cases. The second angle may be between about 0-degrees and 180-degrees, for example, between about 10-degrees and about 170-degrees, about 20-degrees and about 160-degrees, between about 30-degrees and about 150-degrees, about 40-degrees and about 140-degrees, between about 50-degrees and about 130-degrees, about 60-degrees and about 120-degrees, between about 70-degrees and about 110-degrees, about 80-degrees and about 100-degrees, between about 90-degrees and about 90-degrees, or any value or range between any of these values or ranges or any value or range bounded by any combination of these values, although values or ranges outside these values or ranges can be used in some cases. In this way, the connector portion 491 may be off center from the sensor portion 493 due to the differences in length and angles of the IMU and memory portion 492 to each of the connector portion 491 and the sensor portion 493.

The fourth portion 485 may have a height offset 496, for example, from a first height to a second height. The height offset 496 may be linear, having a height and a distance. The height may be between about 0 mm and about 50 mm, for example, between about 5 mm and about 45 mm, between about 10 mm and about 40 mm, between about 15 mm inch and about 35 mm, between about 20 mm and about 30 mm, between about 25 mm and about 25 mm, or any value or range between any of these values or ranges or any value or range bounded by any combination of these values, although values or ranges outside these values or ranges can be used in some cases. The distance may be between about 0 mm and about 50 mm, for example, between about 5 mm and about 45 mm, between about 10 mm and about 40 mm, between about 15 mm inch and about 35 mm, between about 20 mm and about 30 mm, between about 25 mm and about 25 mm, or any value or range between any of these values or ranges or any value or range bounded by any combination of these values, although values or ranges outside these values or ranges can be used in some cases.

FIGS. 5A-5B illustrate an example embodiment of an exciter for the blood pressure monitoring systems disclosed herein. The exciters disclosed in FIGS. 5A-5B may be implemented in connection with the sensors as disclosed herein (for example, the exciters may attach to exciter regions of the sensors 400,450 in FIGS. 4A-4B). FIG. 5A illustrates an exploded view of an example embodiment of an exciter 500. As illustrated in FIG. 5A, the exciter 500 may include a back plate 502, a magnet 504, a top plate 506, a wire coil 508, and a spring housing 510. The exciter 500 may receive an applied signal from a signal generation source. In some cases, the wire coil 508 may receive the applied signal. The applied signal may be an analog (or digital) electrical signal corresponding to a range of frequencies. The wire coil 508 may generate an electric field according to the applied signal. In some cases, the electric field generated by the wire coil 508 may oscillate according to oscillations of the frequencies of the applied signal. The spring housing 510 may include an internal structure configured to allow adjustment of a position of an exciter nob. The internal structure may attach to the wire coil 508, such that the wire coil 508 is wrapped around the internal structure. The attachment of the internal structure and the wire coil 508 form a movable internal component, which may move in response to changes in an electric field generated by the wire coil 508 (and corresponding magnetic force by the magnet 504). In some cases, the magnet 504 may produce a magnetic force in response to the electric field generated by the wire coil 508. The magnet 504 may produce the magnetic force to adjust the movable component, which may contact a spring. For example, the wire coil 508 may generate an electric field corresponding to an applied signal to the sensor, such that the magnet 504 produces a magnetic force in response to the electric field. The movement of the movable component may cause an exciter nob to adjust a position and strike a patient. The exciter nob striking the patient may result in an acoustic signal to travel within the patient that corresponds to the applied signal to the sensor. The movement of the exciter nob may correspond to an emitted signal as disclosed herein. The exciter 500 may include any component, material, feature, capability, function as described with respect to any other exciter herein (such as, exciter 600, 700, and 800).

The back plate 502 may provide structural support to the components of the exciter 500. In some cases, the back plate 502 may attach to the spring housing 510 to form a housing for the exciter 500. In some cases, the back plate 502 may fit within the spring housing 510. In some cases, the back plate 502 may affix around the spring housing 510. In some cases, the back plate 502 may attach to a spring within the housing formed by the back plate 502 and the spring housing 510. In some cases, the spring may suspend from the back plate. The spring may suspend from the back plate 502 and attach to an exciter nob. The exciter nob may oscillate according to an applied signal to the sensor. The back plate 502 may be constructed of various materials. The back plate 502 may include bio-compatible materials. In some cases, the back plate 502 may be metal and/or a metallic alloy. In some cases, the back plate 502 may be magnetic or non-magnetic metal (or metallic alloy). For example, the back plate 502 may be stainless steel, aluminum, copper, titanium, cobalt chrome, magnesium, gold, platinum, silver, iridium, nitinol, and/or tantalum, and/or any other metal (or alloy) suitable for the applications disclosed herein. In some cases, the stainless steel may include 200 series stainless steel, 300 series stainless steel, 400 series stainless steel, duplex steel, 17-4PH, and/or carbon steel. In some cases, the back plate 502 may be rubber material. For example, the back plate 502 may be silicone rubber (such as liquid silicone rubber and/or high consistency rubber). In some cases, the back plate 502 may be plastic. For example, the back plate 502 may be polyethylene, polypropylene, polymethyl methacrylate (PMMA), polyvinyl chloride (PVC), polyamide, acrylonitrile butadiene styrene (ABS), polycarbonate, or any other plastic suitable for the applications disclosed herein.

The magnet 504 may provide magnetic force to the components of the exciter. In some cases, the magnet 504 may produce a magnetic force in response to an electric field generated by the wire coil 508. The magnet 504 may produce a magnetic force to adjust the movable component according to the electric field generated by the wire coil 508. For example, the wire coil 508 may generate an electric field corresponding to an applied signal to the sensor, such that the magnet 504 produces a magnetic force in response to the electric field. The magnet 504 may have predetermined dimensions. The magnet 504 may include an outer diameter, an inner diameter, and a thickness. In some cases, the outer diameter may be any value within, or range between, 0-5 mm, 5-10 mm, 10-15 mm, 15-20 mm, 0-10 mm, 0-15 mm, 0-20 mm, or any individual value or combination of outer diameter lengths applicable. The inner diameter may be any value within, or range between, 0-1 mm, 1-3 mm, 3-5 mm, 5-8 mm, 8-10 mm, or any individual value or combination of outer diameter lengths applicable. The thickness may be any value within, or range between, 0-2 mm, 2-5 mm, 5-10 mm, or any individual value or combination of outer diameter lengths applicable. In some cases, the magnet 504 is in a permanent (or non-permanent) position in the housing established by the back plate 502 and the spring housing 510. The magnet 504 may be constructed of various materials. The magnet 504 may be constructed from metals or metal alloys. For example, the magnet 504 may be constructed from steel, stainless steel, carbon steel, and/or any other magnetic metals (or alloys) for the particular application. In some cases, the stainless steel may include 200 series stainless steel, 300 series stainless steel, 400 series stainless steel, duplex steel, 17-4PH, and/or carbon steel. In some cases, the exciter 500 may include the magnet 504 as part of a voice coil actuator (including a moving coil actuator or a moving magnet actuator) and/or a solenoid.

The top plate 506 may provide separation between the magnet 504 and an exciter nob. The top plate 506 may be constructed of various materials. The top plate 506 may include bio-compatible materials. In some cases, the top plate 506 may be metal and/or a metallic alloy. In some cases, the top plate 506 may be magnetic or non-magnetic metal (or metallic alloy). For example, the top plate 506 may be stainless steel, aluminum, copper, titanium, cobalt chrome, magnesium, gold, platinum, silver, iridium, nitinol, and/or tantalum, and/or any other metal (or alloy) suitable for the applications disclosed herein. In some cases, the stainless steel may include 200 series stainless steel, 300 series stainless steel, 400 series stainless steel, duplex steel, 17-4PH, and/or carbon steel. In some cases, the top plate 506 may be rubber material. For example, the top plate 506 may be silicone rubber (liquid silicone rubber and/or high consistency rubber). In some cases, the top plate 506 may be plastic. For example, the top plate 506 may be polyethylene, polypropylene, PMMA, PVC, polyamide, ABS, polycarbonate, or any other plastic suitable for the applications disclosed herein. The top plate 506 may have predetermined dimensions. The top plate 506 may include an outer diameter, an inner diameter, and a thickness. In some cases, the outer diameter may be any value within, or range between, 0-5 mm, 5-10 mm, 10-15 mm, 15-20 mm, 0-10 mm, 0-15 mm, 0-20 mm, or any individual value or combination of outer diameter lengths applicable. In some cases, the top plate 506 may have an outer diameter greater (or lesser) than the magnet 504. The inner diameter may be any value within, or range between, 0-1 mm, 1-3 mm, 3-5 mm, 5-8 mm, 8-10 mm, or any individual value or combination of outer diameter lengths applicable. In some cases, the top plate 506 may have an inner diameter greater (or lesser) than the magnet 504. The thickness may be any value within, or range between, 0-2 mm, 2-5 mm, 5-10 mm, or any individual value or combination of outer diameter lengths applicable. In some cases, the top plate 506 may have a thickness greater (or lesser) than the magnet 504.

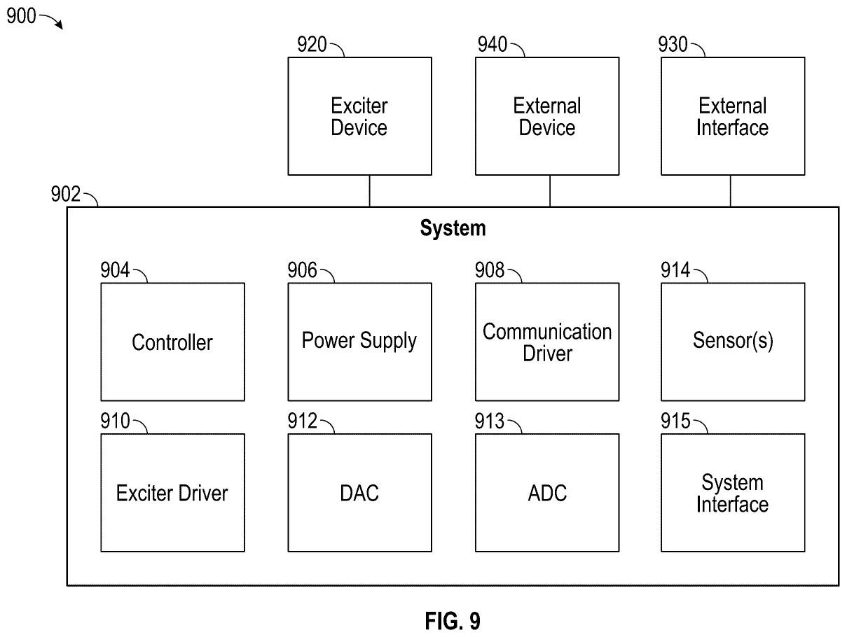

The wire coil 508 may generate an electric field in response to receiving an applied signal. The wire coil 508 may receive the applied signal from a signal generation source. In some cases, the signal generation source is local to the sensor (or external signal generation source coupled to the sensor). The signal generation source may transmit the applied signal from an exciter driver (for example, exciter driver 910 in FIG. 9) to an RD connector of the sensor (for example, connector 416, 466 in FIGS. 4A and 4B). The RD connector may connect to the wire coil 508 to transmit the applied signal. The applied signal may be an analog (or digital) electrical signal corresponding to a range of frequencies. For example, the applied signal may include frequencies 0-10 Hz, 10-100 Hz, 100-1000 Hz, 1000-10000 Hz, 10000-100000 Hz, 100000-1000000 Hz, 0-100 Hz, 0-1000 Hz, 0-10000 Hz, 0-100000 Hz, 0-1000000 Hz, or any individual value or combination of frequencies applicable. The wire coil 508 may generate the electric field according to the applied signal. In some cases, the electric field generated by the wire coil 508 may oscillate according to oscillations of the frequencies. The oscillations of the electric field generated by the wire coil 508 may cause the magnet 504 to produce a magnetic force, which may cause movement of the wire coil 508 (and the movable component). The wire coil 508 may be constructed of various materials. In some cases, the wire coil 508 may be copper (or another conductive material). The wire coil 508 may be constructed of predetermined dimensions. In some cases, the wire coil 508 may include a plurality of layers and a number of turns of a coil. For example, the plurality of layers may be any value within, or range between, 0-2 layers, 2-5 layers, 5-10 layers, 10-20 layers, 20-30 layers, 30-50 layers, 0-5 layers, 0-10 layers, 0-20 layers, 0-30 layers, 0-50 layers, or any individual value or combination of layers applicable. In some examples, the number of turns of the coil may be any value within, or range between, 0-25 turns, 25-50 turns, 50-100 turns, 100-200 turns, 200-500 turns, 500-1000 turns, 0-25 turns, 0-50 turns, 0-100 turns, 0-200 turns, 0-500 turns, 0-1000 turns, or any individual value or combination of turns applicable. The wire coil 508 may include a material thickness. For example, the material thickness may be any value within, or range between, 0-0.01 mm, 0.01-0.05 mm, 0.05-0.10 mm, 0.10-0.25 mm, 0.25-0.50 mm, 0-0.05 mm, 0-0.10 mm, 0-0.25 mm, 0-0.50 mm or any individual value or combination of thicknesses applicable. The wire coil 508 may include an outer diameter, an inner diameter, and a thickness. In some cases, the outer diameter may be any value within, or range between, 0-5 mm, 5-10 mm, 10-15 mm, 15-20 mm, 0-10 mm, 0-15 mm, 0-20 mm, or any individual value or combination of outer diameter lengths applicable. In some cases, the wire coil 508 may have an outer diameter greater (or lesser) than the magnet 504 and/or the top plate 506. The inner diameter may be any value within, or range between, 0-5 mm, 5-10 mm, 10-15 mm, 15-20 mm, 0-10 mm, 0-15 mm, 0-20 mm, or any individual value or combination of outer diameter lengths applicable. In some cases, the wire coil 508 may have an inner diameter greater (or lesser) than the magnet 504 and/or the top plate 506. The thickness may be any value within, or range between, 0-2 mm, 2-5 mm, 5-10 mm, 0-5 mm, 0-10 mm or any individual value or combination of outer diameter lengths applicable. In some cases, the wire coil 508 may have a thickness greater (or lesser) than the magnet 504 and/or the top plate 506. In some cases, the wire coil 508 is in a permanent (or non-permanent) position in the housing established by the back plate 502 and the spring housing 510. In some cases, the exciter 500 may include the wire coil 508 as part of a voice coil actuator (including a moving coil actuator or a moving magnet actuator) and/or a solenoid.

The spring housing 510 may provide structural support to the exciter components. In some cases, the spring housing 510 may attach to the back plate 502 to form a housing for the exciter. In some cases, at least part of the spring housing 510 may fit within the back plate 502. In some cases, at least part of the spring housing 510 may fit around the back plate 502. The spring housing 510 may include an internal structure configured to a adjust position of an exciter nob. The internal structure may attach to the wire coil 508, such that the wire coil 508 is wrapped around the internal structure. The attachment of the internal structure and the wire coil 508 form a movable internal component, which moves in response to changes in an electric field generated by the wire coil 508 (and corresponding magnetic force by the magnet 504). The spring housing 510 may be constructed of various materials. The spring housing 510 may include bio-compatible materials. In some cases, the spring housing 510 may be metal and/or a metallic alloy. In some cases, the spring housing 510 may be magnetic or non-magnetic metal (or metallic alloy). For example, the spring housing 510 may be stainless steel, aluminum, copper, titanium, cobalt chrome, magnesium, gold, platinum, silver, iridium, nitinol, and/or tantalum, and/or any other metal (or alloy) suitable for the applications disclosed herein. In some cases, the stainless steel may include 200 series stainless steel, 300 series stainless steel, 400 series stainless steel, duplex steel, 17-4PH, and/or carbon steel. In some cases, the spring housing 510 may be rubber material. For example, the spring housing 510 may be silicone rubber (liquid silicone rubber and/or high consistency rubber). In some cases, the spring housing 510 may be plastic. For example, the spring housing 510 may be polyethylene, polypropylene, PMMA, PVC, polyamide, ABS, polycarbonate, or any other plastic suitable for the applications disclosed herein.

FIG. 5B illustrates a cross-sectional view of an assembled exciter 550. In some instances, the assembled exciter 550 may include the elements discussed in FIG. 5A, a suspension spring 552, an exciter nob 554, and a base plate 560. In some cases, the assembled exciter 550 may contact a patient's skin to apply a signal (such as an acoustic signal caused by the exciter nob 554) for measuring physiological parameters of the patient in a continuous and non-invasive manner (such as measuring blood pressure). The suspension spring 552 may attach to the back plate 502 and the exciter nob 554. In some examples, the suspension spring 552 may attach to the back plate 502 via a soldering method, mechanical crimping, or another method of attaching the suspension spring 552 to the back plate 502. In some cases, the suspension spring 552 may attach to the exciter nob 554 via a soldering method, mechanical crimping, or another method of attaching the suspension spring 552 to the exciter nob 554.

In some instances, the suspension spring 552 may oscillate in response to an applied signal. The suspension spring 552 may adjust position according to a state of the wire coil 508. When the wire coil 508 is in a non-driven state, the suspension spring 552 may apply mechanical force to the exciter nob 554 towards the back plate 502 such that the exciter nob 554 is mechanically stopped from moving towards the back plate 502 by the spring housing 510 and the top plate 506. When the wire coil 508 is in a driven state, the suspension spring 552 may extend and compress such that the internal structure of the spring housing 510, the wire coil 508, and the exciter nob 554 oscillate from a minimum position to a maximum position. In some cases, the minimum position may be when the spring housing 510, the wire coil 508, and the exciter nob 554 are closest to the top plate 506. In some cases, the maximum position may be when the spring housing 510, the wire coil 508, and the exciter nob 554 are farthest from the top plate 506. The range between the minimum position and the maximum position may correspond to an amplitude of an applied signal, which may cause the wire coil 508 to generate an electric field and the magnet 504 to produce magnetic force. In some cases, the suspension spring 552 may be a compression spring, a tension spring, constant force spring, spring-powered reel, or a torsion spring.

In some cases, the suspension spring 552 may be constructed of various materials. For example, the suspension spring 552 may be stainless steel, hard-drawn wire, elgiloy, bronze, nickel, chrome, silver, platinum (including platinum-iridium and platinum-tungsten), titanium, and/or any other metal (or alloy) suitable for the applications disclosed herein. In some cases, the stainless steel may include 200 series stainless steel, 300 series stainless steel (including 316 grade), 400 series stainless steel, duplex steel, 17-4PH, and/or carbon steel. In some cases, the suspension spring 552 may include predetermined dimensions. In some cases, the suspension spring 552 may include a plurality of layers and a number of turns of a coil. For example, the plurality of layers may be any value within, or range between, 0-2 layers, 2-5 layers, 5-10 layers, 10-20 layers, 20-50 layers, 50-100 layers, 0-5 layers, 0-10 layers, 0-20, 0-50, 0-100 layers, or any individual value or combination of layers applicable. In some examples, the number of turns of the coil may be any value within, or range between, 0-25 turns, 25-50 turns, 50-100 turns, 100-200 turns, 200-500 turns, 500-1000 turns, 0-25 turns, 0-50 turns, 0-100 turns, 0-200 turns, 0-500 turns, 0-1000 turns, or any individual value or combination of turns applicable. The suspension spring 552 may include a material thickness. For example, the material thickness may be any value within, or range between, 0-0.01 mm, 0.01-0.05 mm, 0.05-0.10 mm, 0.10-0.25 mm, 0.25-0.50 mm, 0-0.05 mm, 0-0.10 mm, 0-0.25 mm, 0-0.50 mm or any individual value or combination of thicknesses applicable. The suspension spring 552 may include an outer diameter, an inner diameter, and a length. In some cases, the outer diameter may be any value within, or range between, 0-5 mm, 5-10 mm, 10-15 mm, 15-20 mm, 0-10 mm, 0-15 mm, 0-20 mm, or any individual value or combination of outer diameter lengths applicable. In some cases, the suspension spring 552 may have an outer diameter greater (or lesser) than the magnet 504 and/or the top plate 506. The inner diameter may be any value within, or range between, 0-5 mm, 5-10 mm, 10-15 mm, 15-20 mm, 0-10 mm, 0-15 mm, 0-20 mm, or any individual value or combination of outer diameter lengths applicable. In some cases, the suspension spring 552 may have an inner diameter greater (or lesser) than the magnet 504 and/or the top plate 506. In some cases, the outer diameter of the suspension spring 552 may be lesser (or greater) than the inside diameter of the magnet 504 and/or the top plate 506. The thickness may be any value within, or range between, 0-2 mm, 2-5 mm, 5-10 mm, 0-5 mm, 0-10 mm or any individual value or combination of outer diameter lengths applicable. In some cases, the suspension spring 552 may have a thickness greater (or lesser) than the magnet 504 and/or the top plate 506.

The exciter nob 554 may extend and retract in response to an applied signal. The exciter nob 554 may attach to the suspension spring 552. The exciter nob 554 may include alignment components 556 and a central extension component 558. The exciter nob 554 may attach to the suspension spring 552, such that the suspension spring 552 wraps around the central extension component 558. In some cases, the central extension component 558 mechanically affixes to the suspension spring 552. The alignment components 556 may fit within an opening of the internal structure of the spring housing 510. The alignment components 556 may position the exciter nob 554 to align with an opening of the base plate 560. The alignment components 556 may be positioned inside of a maximum outside diameter of the exciter nob 554. The alignment components 556 may be positioned to be within the inner diameter of the magnet 504 and/or the top plate 506. The exciter nob 554 may be constructed with various materials. The exciter nob 554 may include bio-compatible materials. In some cases, the exciter nob 554 may be rubber material. For example, the exciter nob 554 may be silicone rubber (liquid silicone rubber and/or high consistency rubber). In some cases, the exciter nob 554 may be metal and/or a metallic alloy. In some cases, the exciter nob 554 may be magnetic or non-magnetic metal (or metallic alloy). For example, the exciter nob 554 may be stainless steel, aluminum, copper, titanium, cobalt chrome, magnesium, gold, platinum, silver, iridium, nitinol, and/or tantalum, and/or any other metal (or alloy) suitable for the applications disclosed herein. In some cases, the stainless steel may include 200 series stainless steel, 300 series stainless steel, 400 series stainless steel, duplex steel, 17-4PH, and/or carbon steel. In some cases, the exciter nob 554 may be plastic. For example, the exciter nob 554 may be polyethylene, polypropylene, PMMA, PVC, polyamide, ABS, polycarbonate, or any other plastic suitable for the applications disclosed herein. The exciter nob 554 may be constructed with predetermined dimensions. For example, the maximum outside diameter of the exciter nob 554 may be greater (or lesser) than the inside diameter of the magnet 504 and/or the top plate 506. In some cases, the maximum outside diameter of the exciter nob 554 may be lesser (or greater) than the opening of the base plate 560.

The base plate 560 may provide the opening for the exciter nob 554 to contact the patient's skin. The base plate 560 may be positioned to contact the patient. The opening of the base plate 560 may be wide enough to give clearance to the exciter nob 554 as the exciter nob 554 extends and retracts. The base plate 560 may be constructed of various materials. The base plate 560 may include bio-compatible materials. In some cases, the base plate 560 may be metal and/or a metallic alloy. In some cases, the base plate 560 may be magnetic or non-magnetic metal (or metallic alloy). For example, the base plate 560 may be stainless steel, aluminum, copper, titanium, cobalt chrome, magnesium, gold, platinum, silver, iridium, nitinol, and/or tantalum, and/or any other metal (or alloy) suitable for the applications disclosed herein. In some cases, the stainless steel may include 200 series stainless steel, 300 series stainless steel, 400 series stainless steel, duplex steel, 17-4PH, and/or carbon steel. In some cases, the base plate 560 may be rubber material. For example, the base plate 560 may be silicone rubber (liquid silicone rubber and/or high consistency rubber). In some cases, the base plate 560 may be plastic. For example, the base plate 560 may be polyethylene, polypropylene, PMMA, PVC, polyamide, ABS, polycarbonate, or any other plastic suitable for the applications disclosed herein.

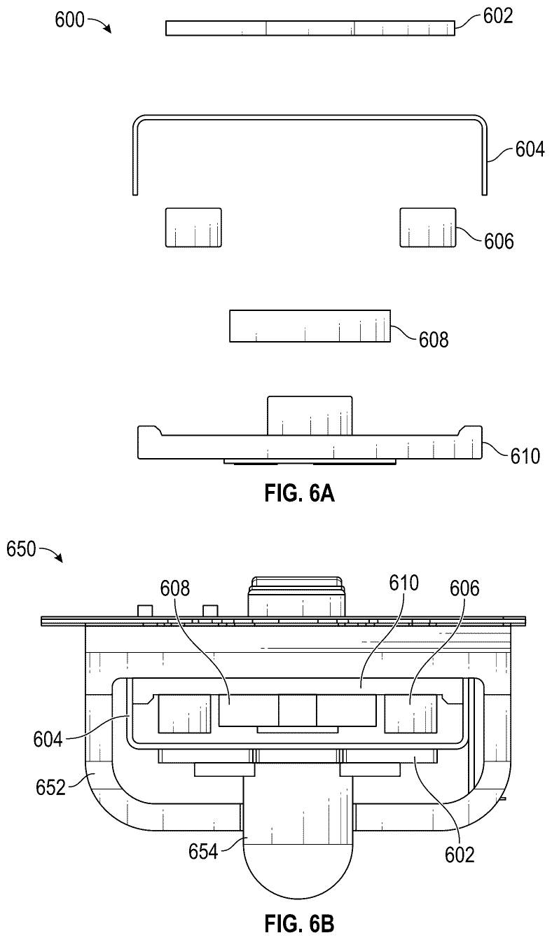

FIGS. 6A-6B illustrate an example embodiment of an exciter 600 for the sensors disclosed herein. The exciters disclosed in FIGS. 6A-6B may be implemented in the sensors as disclosed herein (for example, the exciters may attach to exciter regions of the sensors 400,450 in FIGS. 4A-4B). FIG. 6A illustrates an exploded view of an example embodiment of an exciter 600. As illustrated in FIG. 6A, the exciter 600 may include a top plate 602, suspension spring 604, magnet 606, wire coil 608, and a back plate 610. The exciter 600 may receive an applied signal from a signal generation source. In some cases, the wire coil 608 may receive the applied signal. The applied signal may be an analog (or digital) electrical signal corresponding to a range of frequencies. The wire coil 608 may generate an electric field according to the applied signal. In some cases, the electric field generated by the wire coil 608 may oscillate according to oscillations of the frequencies of the applied signal. In some cases, the magnet 606 may produce a magnetic force in response to an electric field generated by the wire coil 608. The magnet 606 may produce a magnetic force to adjust a position of the top plate 602 according to the electric field generated by the wire coil 608. For example, the wire coil 608 may generate an electric field corresponding to an applied signal to the sensor, such that the magnet 606 produces a magnetic force in response to the electric field. The suspension spring 604 may attach to the top plate 602, which may be magnetic and operate to adjust in response to the magnetic field. The top plate 602 may attach to the exciter nob, such that the magnetic force may cause movement of the top plate 602 and the suspension spring 604 (such as an oscillation) and may cause the exciter nob to oscillate which may cause the exciter nob to strike a patient. The exciter 600 may include any component, material, feature, capability, function as described with respect to any other exciter herein (such as, exciter 500, 700, and 800).

The top plate 602 may provide separation between the magnet 606 and an exciter nob. The top plate 602 may be constructed of various materials as described herein (such as the materials disclosed for the top plate 506 in FIGS. 5A-5B). The top plate 602 may be constructed of predetermined dimensions as described herein (for example, the dimensions disclosed for the top plate 506 in FIGS. 5A-5B). The top plate 602 may include an outer diameter, an inner diameter, and a thickness as described herein (for example, the outer diameter, inner diameter, and thickness disclosed for the top plate 506 in FIGS. 5A-5B).

The suspension spring 604 may oscillate in response to an applied signal. The suspension spring 604 may adjust position according to a state of the wire coil 608. When the wire coil 608 is in a non-driven state, the suspension spring 604 may apply mechanical force to the exciter nob towards the back plate 610 such that the exciter nob is mechanically stopped from moving towards the back plate 610 by the top plate 602. When the wire coil 608 is in a driven state, the suspension spring 604 may extend and compress such that the top plate 602 and the exciter nob oscillate from a minimum position to a maximum position. In some cases, the minimum position may be when the top plate 602 and the exciter nob are closest to the back plate 610. In some cases, the maximum position may be when the top plate 602 and the exciter nob are farthest from the back plate 610. The range between the minimum position and the maximum position may correspond to an amplitude of an applied signal, which may cause the wire coil 608 to generate an electric field and the magnet 606 to produce magnetic force. In some cases, the suspension spring 604 may be a compression spring, a tension spring, constant force spring, spring-powered reel, or a torsion spring. In some cases, the suspension spring 604 may be constructed of various materials (for example, the materials disclosed for the suspension spring 552 in FIG. 5B). In some cases, the suspension spring 604 may include predetermined dimensions (for example, the layers, turns, material thickness, outer diameter, inner diameter, and thickness disclosed for the suspension spring 552 in FIG. 5B).

The magnet 606 may provide magnetic force to the components of the exciter 600. In some cases, the magnet 606 may produce a magnetic force in response to an electric field generated by the wire coil 608. The magnet 606 may produce a magnetic force to oscillate a spring according to the electric field generated by the wire coil 608. For example, the wire coil 608 may generate an electric field corresponding to an applied signal to the sensor, such that the magnet 606 produces a magnetic force in response to the electric field. The magnetic force may cause movement of the top plate 602 and the suspension spring 604 (such as an oscillation). The movement of the suspension spring 604 may cause an exciter nob to oscillate which may cause the exciter nob to strike a patient. The exciter nob striking the patient may result in an acoustic signal to travel within the patient that corresponds to the applied signal to the sensor. The magnet 606 may have predetermined dimensions (for example, the outer diameter, inner diameter, and thickness disclosed for the magnet 504 in FIGS. 5A-5B). The magnet 606 may be constructed of various materials (for example, the materials disclosed for the magnet 504 in FIGS. 5A-5B). In some cases, the magnet 606 is in a permanent (or non-permanent) position in a housing of the exciter 600. In some cases, the exciter 600 may include the magnet 606 as part of a voice coil actuator (including a moving coil actuator or a moving magnet actuator) and/or a solenoid.

The wire coil 608 may generate an electric field in response to receiving an applied signal. The wire coil 608 may receive the applied signal as an analog (or digital) electrical signal corresponding to a range of frequencies as described herein (for example, the range of frequencies disclosed for the wire coil 508 in FIGS. 5A-5B). The wire coil 608 may be constructed of various materials as described herein (for example, the materials disclosed for the wire coil 508 in FIGS. 5A-5B). The wire coil 608 may be constructed of predetermined dimensions as described herein (for example, the dimensions disclosed for the wire coil 508 in FIGS. 5A-5B). The wire coil 608 may include a material thickness as described herein (for example, the material thickness disclosed for the wire coil 508 in FIGS. 5A-5B). The wire coil 608 may include an outer diameter, an inner diameter, and a thickness as described herein (for example, the outer diameter, inner diameter, and thickness disclosed for the wire coil 508 in FIGS. 5A-5B). In some cases, the outer diameter of the wire coil 608 may be lesser (or greater) than the inner diameter of the magnet 606. For example, the wire coil 608 may be positioned within the inner diameter of the magnet 606. In some cases, the wire coil 608 is in a permanent (or non-permanent) position in a housing. In some cases, the exciter 600 may include the wire coil 608 as part of a voice coil actuator (including a moving coil actuator or a moving magnet actuator) and/or a solenoid.

The back plate 610 may provide structural support to the exciter components. In some cases, the back plate 610 may fit within a housing of the exciter 600. In some cases, the suspension spring 604 may suspend from the back plate 610. The suspension spring 604 may suspend from the back plate 610 and attach to an exciter nob. The exciter nob may oscillate according to an applied signal to the sensor. The back plate 610 may be constructed of various materials (for example, the materials disclosed for the back plate 502 in FIGS. 5A-5B).

FIG. 6B illustrates a cross-sectional view of the exciter assembled 650. In some instances, the assembled exciter 650 may include the elements discussed in FIG. 6A, a housing 652, and an exciter nob 654. In some cases, the assembled exciter 650 may contact a patient's skin to apply an acoustic signal (from the exciter nob 654) for measuring physiological parameters of the patient in a continuous and non-invasive manner (for example, measuring blood pressure).

The housing 652 may provide structural support to the exciter components. In some cases, the housing 652 may attach to the back plate 610 and the suspension spring 604. The housing 652 may encompass the back plate 610 and the suspension spring 604. The housing 652 may be positioned to contact the patient. An opening of the housing 652 may be wide enough to give clearance to the exciter nob 654 as the suspension spring 604 extends and retracts. The housing 652 may be constructed of various materials. The housing 652 may include bio-compatible materials. In some cases, the housing 652 may be metal and/or a metallic alloy. In some cases, the housing 652 may be magnetic or non-magnetic metal (or metallic alloy). For example, the housing 652 may be stainless steel, aluminum, copper, titanium, cobalt chrome, magnesium, gold, platinum, silver, iridium, nitinol, and/or tantalum, and/or any other metal (or alloy) suitable for the applications disclosed herein. In some cases, the stainless steel may include 200 series stainless steel, 300 series stainless steel, 400 series stainless steel, duplex steel, 17-4PH, and/or carbon steel. In some cases, the housing 652 may be rubber material. For example, the housing 652 may be silicone rubber (liquid silicone rubber and/or high consistency rubber). In some cases, the housing 652 may be plastic. For example, the housing 652 may be polyethylene, polypropylene, PMMA, PVC, polyamide, ABS, polycarbonate, or any other plastic suitable for the applications disclosed herein.

The exciter nob 654 may adjust a position in response to extension and contraction of the suspension spring 604. The exciter nob 654 may attach to the suspension spring 604. The exciter nob 654 may attach to the top plate 602 and the suspension spring 604, such that the top plate 602 forms a base of the exciter nob 654. The exciter nob 654 may be positioned to be within the inner diameter of the magnet 606 and/or the wire coil 608. The exciter nob 654 may be constructed with various materials (for example, the materials disclosed for the exciter nob 554 in FIG. 5B).

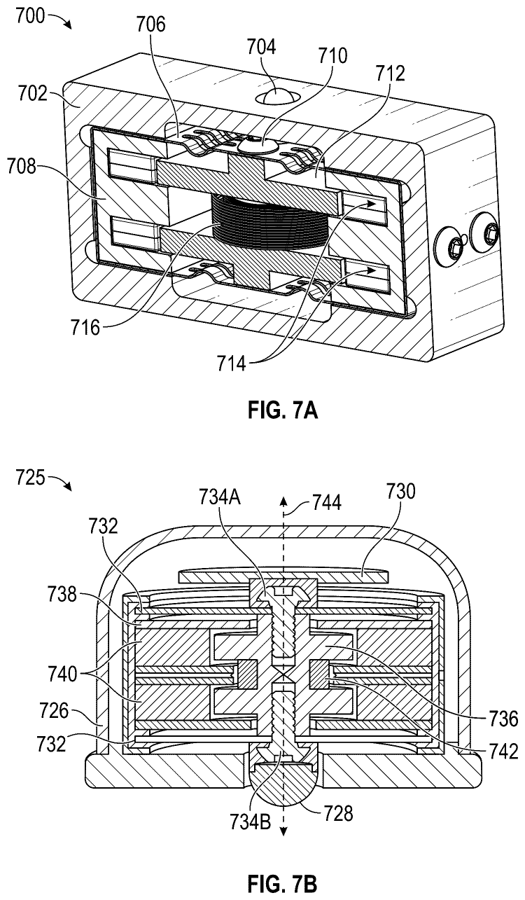

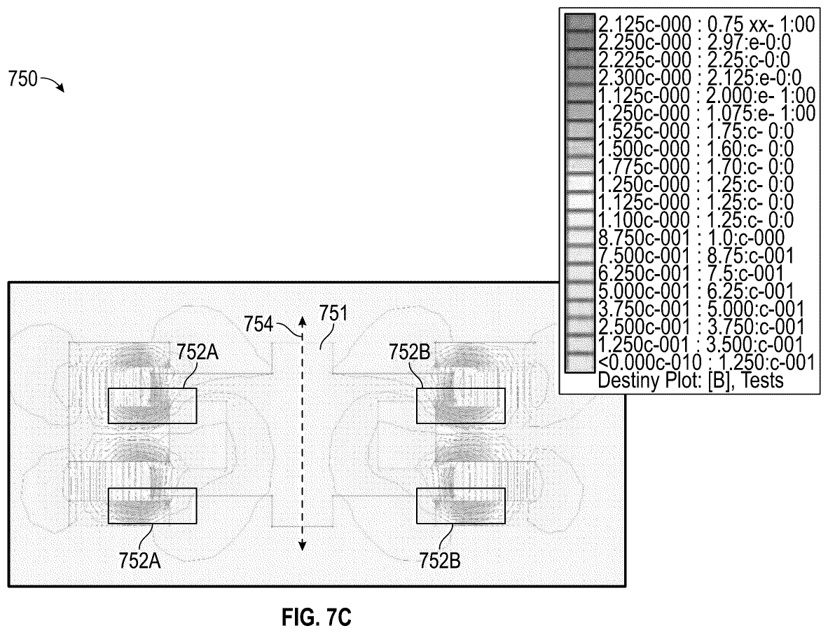

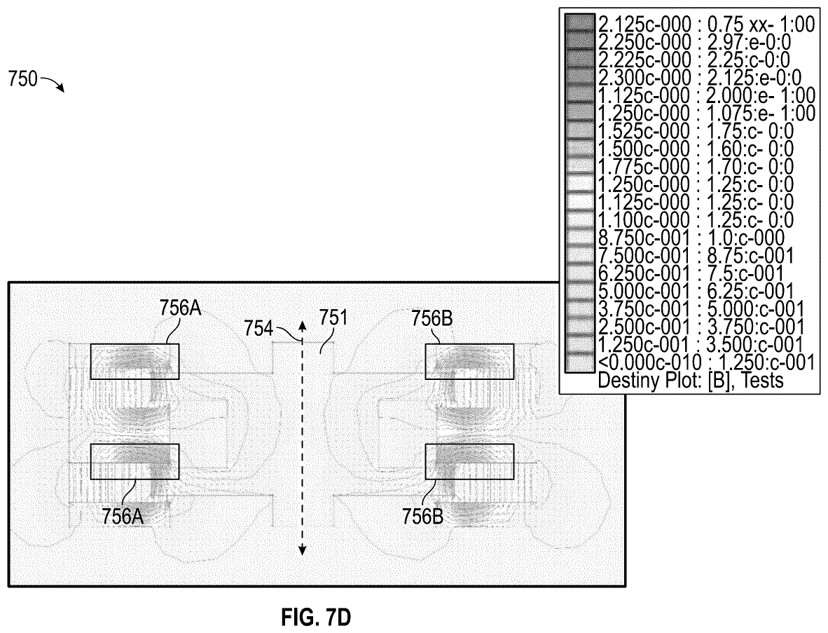

FIGS. 7A-7D illustrate example embodiments of an exciter for the sensors disclosed herein and graphs of magnetic characteristics for the exciter. The exciters disclosed in FIGS. 7A-7B may be implemented in the sensors as disclosed herein (for example, the exciters may couple to exciter regions of the sensors 400,450 in FIGS. 4A-4B). FIG. 7A illustrates a perspective view of an example embodiment of the exciter 700. As illustrated in FIG. 7A, the exciter 700 may include a housing 702, an exciter nob 704, a suspension spring 706, an internal structure 708, a fastener 710, a movable component 712, magnets 714, and a wire coil 716. The exciter 700 may receive a signal from a signal generation source. In some cases, the wire coil 716 may receive the signal. The signal may be an analog (or digital) electrical signal corresponding to a range of frequencies. The wire coil 716 may generate an electric field according to the signal. In some cases, the electric field generated by the wire coil 716 may oscillate according to oscillations of the frequencies of the signal. The movable component 712 may adjust a position of the exciter nob 704. The movable component 712 may attach to the wire coil 716, such that the wire coil 716 is wrapped around part of the movable component 712. In some cases, the magnets 714 may produce a magnetic force in response to an electric field generated by the wire coil 716. The magnets 714 may produce a magnetic force to adjust a position of the movable component 712 and the wire coil 716, which may cause the suspension spring 706 to adjust position. For example, the wire coil 716 may generate an electric field corresponding to an applied signal to the sensor, such that the magnets 714 produce a magnetic force in response to the electric field. The movement of the movable component 712, the wire coil 716, and/or the suspension spring 706 may cause the exciter nob 704 to oscillate, causing the exciter nob 704 to strike a patient. In some instances, the movement of the suspension spring 706 may cause the exciter nob 704 to oscillate which may cause the exciter nob 704 to strike a patient. Accordingly, a sensor (such as, those described herein) may receive the signal (for example, via detectors) after the signal passes through the patient's body. The exciter 700 may include any component, material, feature, capability, function as described with respect to any other exciter herein (such as, exciter 500, 600, and 800).

The housing 702 may provide structural support to the exciter components. In some cases, the housing 702 may provide an opening for the exciter nob 704. The housing 702 may be positioned to contact the patient. An opening of the housing 702 may be wide enough to give clearance to the exciter nob 704 as the suspension spring 706 extends and retracts. The housing 702 may be constructed of various materials, for example, including bio-compatible materials, metal, and/or a metallic alloy. In some cases, the housing 702 may be magnetic or non-magnetic metal (or metallic alloy). For example, the housing 702 may be stainless steel, aluminum, copper, titanium, cobalt chrome, magnesium, gold, platinum, silver, iridium, nitinol, and/or tantalum, and/or any other metal (or alloy) suitable for the applications disclosed herein. In some cases, the stainless steel may include 200 series stainless steel, 300 series stainless steel, 400 series stainless steel, duplex steel, 17-4PH, and/or carbon steel. In some cases, the housing 702 may be rubber material. For example, the housing 702 may be silicone rubber (liquid silicone rubber and/or high consistency rubber). In some cases, the housing 702 may be plastic. For example, the housing 702 may be polyethylene, polypropylene, PMMA, PVC, polyamide, ABS, polycarbonate, or any other plastic suitable for the applications disclosed herein.

The exciter nob 704 may adjust a position in response to extension and contraction of the suspension spring 706. The exciter nob 704 may attach to the suspension spring 706 via a fastener (such as, fastener 710). The exciter nob 704 may attach to the movable component 712 and the suspension spring 706, such that the movable component 712 forms a base for the exciter nob 704. The exciter nob 704 may be constructed with various materials (for example, the materials disclosed for the exciter nob 554 in FIG. 5B).

The suspension spring 706 may oscillate in response to a signal. The suspension spring 706 may adjust position according to a state of the wire coil 716, such as driven and non-driven states corresponding to a signal applied or not applied to the wire coil 716, respectively. When the wire coil 716 is in a non-driven state, the suspension spring 706 does not move, thus the exciter nob 704 remains in place. When the wire coil 716 is in a driven state, the suspension spring 706 may flex (for example, extending along a central axis aligned with the motion of the exciter nob 704) such that the movable component 712 oscillates from a minimum position to a maximum position. In some cases, the minimum position may be when part of the movable component 712 coupled to the exciter nob 704 is farthest from the housing 702. In some cases, the maximum position may be when part of the movable component 712 coupled to the exciter nob 704 is closest to the housing 702. The range between the minimum position and the maximum position may correspond to an amplitude of an applied signal, which may cause the wire coil 716 to generate an electric field and the magnets 714 to produce magnetic force. In some cases, the suspension spring 706 may be a compression spring, a tension spring, constant force spring, spring-powered reel, or a torsion spring. In some cases, the suspension spring 706 may be constructed of various materials (for example, the materials disclosed for the suspension spring 552 in FIG. 5B). In some cases, the suspension spring 706 may include predetermined dimensions (for example, the layers, turns, material thickness, outer diameter, inner diameter, and thickness disclosed for the suspension spring 552 in FIG. 5B).

The internal structure 708 may provide separation at least between the suspension spring 706 and the magnets 714. The internal structure 708 may include components such as washers, spacers, and/or the like. The internal structure 708 may be constructed of various materials as described herein (for example, the materials disclosed for the back plate 502 and the top plate 506 in FIGS. 5A-5B). The internal structure 708 may include openings to house the magnets 714. The openings may accept surfaces of the movable components 712. In some examples, the internal structure 708 may include at least two components. Each of the two components may be shaped to form two “E”s facing one another.

The fastener 710 may attach the suspension spring 706 to the movable component 712. In some instances, the fastener 710 may attach the exciter nob 704 to the suspension spring 706 and the movable component 712. In some examples, the fastener 710 may include a screw, a rivet, and/or snap fixture, or another type of fastener applicable herein.

The movable component 712 may adjust a position in response to changes in an electric field generated by the wire coil 716 (and corresponding magnetic force by the magnets 714). The movable component 712 may be constructed of various materials as described herein (for example, the materials disclosed for the back plate 502 and the top plate 506 in FIGS. 5A-5B). The movable component 712 may include rectangular parallel surfaces crossed with a rectangular perpendicular surface connecting the parallel surfaces. In some examples, the movable component 712 forms two plus signs stacked on top of each other. Ends of the parallel surfaces may be adjacent to the magnets 714. Both ends of the perpendicular surface may be attached to the suspension spring 706. In some cases, one end of the perpendicular surface may be attached to the exciter nob 704 and the fastener 710. In this manner, the perpendicular surface may mechanically drive the exciter nob 704.

The magnets 714 may provide magnetic force to the components of the exciter 700. In some cases, the magnets 714 may produce a magnetic force in response to an electric field generated by the wire coil 716. The magnets 714 may produce a magnetic force to oscillate a spring according to the electric field generated by the wire coil 716. For example, the wire coil 716 may generate an electric field corresponding to an applied signal to the sensor, such that the magnets 714 produce magnetic forces in response to the electric field. The magnetic forces may interact causing movement of the movable component 712 (such as an oscillation). For example, the magnets 714 may include a first magnet with a north-south configuration and a second magnet with a south-north configuration, such that the placement of the magnets in the exciter 700 repel each other. In this way, when the wire coil 716 generates an electric field, the magnets 714 may each respectively generate a magnetic field with a force corresponding to the electric field from the wire coil 716. Accordingly, the magnetic fields may repel one another with varying strength, causing the movable component 712 to adjust a position corresponding to the strength of the magnetic fields. The movement of the movable component 712 may cause the exciter nob 704 to oscillate which may cause the exciter nob 704 to strike a patient. The exciter nob 704 striking the patient may result in an acoustic signal to travel within the patient that corresponds to the signal, and accordingly, received by the sensor. In some cases, the magnets 714 may include at least two magnets. For example, the magnets 714 may include four sets of magnets. Each set of magnets may include a pair of magnets having opposite (or the same) polarity. In some cases, the magnets 714 may be rectangular. The magnets 714 may have predetermined dimensions. In some cases, the length and/or the width of the magnets 714 may be any value within, or range between, 0-5 mm, 5-10 mm, 10-15 mm, 15-20 mm, 0-10 mm, 0-15 mm, 0-20 mm, or any individual value or combination of outer diameter lengths applicable. The thickness of the magnets 714 may include measurements as disclosed for the magnet 504 in FIGS. 5A-5B. The magnets 714 may be constructed of various materials (for example, the materials disclosed for the magnet 504 in FIGS. 5A-5B). In some cases, the magnets 714 may be in a permanent (or non-permanent) position in a housing of the exciter 700. In some cases, the exciter 700 may include the magnets 714 as part of a voice coil actuator (including a moving coil actuator or a moving magnet actuator) and/or a solenoid.

The wire coil 716 may generate an electric field in response to receiving a signal. The wire coil 716 may receive the signal as an analog (or digital) electrical signal corresponding to a range of frequencies as described herein (for example, the range of frequencies disclosed for the wire coil 508 in FIGS. 5A-5B). The wire coil 716 may be constructed of various materials as described herein (for example, the materials disclosed for the wire coil 508 in FIGS. 5A-5B). The wire coil 716 may be constructed of predetermined dimensions as described herein (for example, the dimensions disclosed for the wire coil 508 in FIGS. 5A-5B). The wire coil 716 may include a material thickness as described herein (for example, the material thickness disclosed for the wire coil 508 in FIGS. 5A-5B). The wire coil 716 may include an outer diameter, an inner diameter, and a thickness as described herein (for example, the outer diameter, inner diameter, and thickness disclosed for the wire coil 508 in FIGS. 5A-5B). In some cases, the outer diameter of the wire coil 716 may be lesser (or greater) than a width of the movable component 712. For example, the wire coil 716 may be wrapped around a central region of the movable component 712 that is narrower than the widest portion of the movable component. In some cases, the wire coil 716 is in a permanent (or non-permanent) position in a housing. In some cases, the exciter 700 may include the wire coil 716 as part of a voice coil actuator (including a moving coil actuator or a moving magnet actuator) and/or a solenoid.

FIG. 7B illustrates a perspective view of an example embodiment of the exciter 725. As illustrated in FIG. 7B, the exciter 725 may include a housing 726, an exciter nob 728, a mass 730, a suspension spring 732, fasteners 734A,B, a movable component 736, an internal structure 738, magnets 740, and a wire coil 742. The exciter 725 may receive an signal from a signal generation source. In some cases, the signal generation source is local to the sensor (or external signal generation source coupled to the sensor). The signal generation source may transmit the signal from an exciter driver (such as exciter driver 910 in FIG. 9) to an RD connector of the sensor (such as connector 416, 466 in FIGS. 4A and 4B). The RD connector may connect and transmit the signal to the wire coil 742. The signal may be an analog (or digital) electrical signal corresponding to a range of frequencies. The wire coil 742 may generate an electric field according to the signal. In some cases, the electric field generated by the wire coil 742 may oscillate according to oscillations of the frequencies of the signal. The movable component 736 may adjust position of the exciter nob 728 and the mass 730. The movable component 736 may attach to the wire coil 742, such that the wire coil 742 is wrapped around part of the movable component 736. The attachment of the movable component 736 and the wire coil 742 may cause movement of the movable component 736 and the wire coil 742 in response to changes in an electric field generated by the wire coil 742 (and corresponding magnetic force by the magnets 740). In some cases, the magnets 740 may produce a magnetic force in response to an electric field generated by the wire coil 742. The magnets 740 may produce a magnetic force to oscillate the movable component 736 and the wire coil 742, which may cause the suspension spring 732 to extend and contract. For example, the wire coil 742 may generate an electric field corresponding to an signal to the sensor, such that the magnets 740 produce a magnetic force in response to the electric field. The movement of the movable component 736, the wire coil 742, and/or the suspension spring 732 may cause the exciter nob 728 and the mass 730 to oscillate, causing the exciter nob 728 to strike a patient. In some instances, the movement of the suspension spring 732 may cause the exciter nob 728 and a mass 730 to oscillate which may cause the exciter nob 728 to strike a patient. Accordingly, a sensor (such as, those described herein) may receive the signal (for example, via detectors) after the signal passes through the patient's body.

The housing 726 may provide structural support to the exciter components. In some cases, the housing 702 may provide an opening for the exciter nob 704. The housing 702 may be positioned to contact the patient. An opening of the housing 702 may be wide enough to give clearance to the exciter nob 704 as the suspension spring 706 extends and retracts. The housing 702 may be constructed of various materials (for example, the materials disclosed for the housing 702 in FIG. 7A).

The exciter nob 728 may adjust a position in response to extension and contraction of the suspension spring 732. The exciter nob 728 may attach to the suspension spring 732 via one of the fasteners 734A,B. The exciter nob 728 may attach to the movable component 736 and the suspension spring 732, such that the movable component 736 and the suspension spring 732 form a base for the exciter nob 728. The exciter nob 728 may be constructed with various materials (for example, the materials disclosed for the exciter nob 554 in FIG. 5B).

The mass 730 may cause adjustment of a position of the exciter nob 728 in response to extension and contraction of the suspension spring 732. The mass 730 may couple to a sensor (for example, sensor 400, 450 as disclosed herein) and provide a force towards the body of the patient (for example, due to gravity). In some cases, when the sensor receives a signal, a wire connector (such as, wire connector 816) may receive the signal to apply to the wire coil 742. The wire coil 742 then generates an electric field, causing adjustment to magnetic field strength of the magnets 740. The magnetic field may cause the moveable component 736 to adjust a position. Accordingly, the signal may cause the movement of the exciter nob 728.

The suspension spring 732 may oscillate in response to an signal. The suspension spring 732 may adjust position according to a state of the wire coil 742, such as driven and non-driven states corresponding to a signal applied or not applied to the wire coil 716, respectively. When the wire coil 742 is in a non-driven state, the suspension spring 732 does not move, thus the exciter nob 704 remains in place. When the wire coil 742 is in a driven state, the suspension spring 732 may flex (for example, extending along the axis 744) such that the movable component 736 oscillates from a minimum position to a maximum position. In some cases, the minimum position may be when the mass 730 is closest to the housing 726 along the axis 744. In some cases, the maximum position may be when the mass 730 is farthest from the housing 726 along the axis 744. The range between the minimum position and the maximum position may correspond to an amplitude of an signal, which may cause the wire coil 742 to generate an electric field and the magnets 740 to produce magnetic force. In some cases, the suspension spring 732 may include one or more spring. The suspension spring 732 may be a compression spring, a tension spring, constant force spring, spring-powered reel, or a torsion spring. In some cases, the suspension spring 732 may be constructed of various materials (for example, the materials disclosed for the suspension spring 552 in FIG. 5B). In some cases, the suspension spring 732 may include predetermined dimensions (for example, the layers, turns, material thickness, outer diameter, inner diameter, and thickness disclosed for the suspension spring 552 in FIG. 5B).

The fasteners 734A,B may attach the suspension spring 732 to the movable component 736. In some instances, the fasteners 734A,B may attach the exciter nob 728 and the mass 730 to the suspension spring 732 and the movable component 736. In some examples, the fasteners 734A,B may include a screw, a rivet, and/or snap fixture, or another fastener applicable herein.

The movable component 736 may adjust a position in response to changes in an electric field generated by the wire coil 742 (and corresponding magnetic force by the magnets 740). The movable component 736 may be constructed of various materials as described herein (for example, the materials disclosed for the back plate 502 and the top plate 506 in FIGS. 5A-5B). The movable component 736 may include rectangular parallel surfaces crossed with a rectangular perpendicular surface connecting the parallel surfaces. In some examples, the movable component 736 forms two plus signs stacked on top of each other. Ends of the parallel surfaces may be adjacent to the magnets 740. Both ends of the perpendicular surface may be attached to the suspension spring 732. In some cases, one end of the perpendicular surface may be attached to the exciter nob 728, the mass 730, and the fasteners 734A,B. In this manner, the perpendicular surface may mechanically drive the exciter nob 728 and provide the same movement for the mass 730.

The internal structure 738 may provide separation at least between the suspension spring 732 and the magnets 740. The internal structure 708 may include components such as washers, spacers, and/or the like. The internal structure 738 may be constructed of various materials as described herein (for example, the materials disclosed for the back plate 502 and the top plate 506 in FIGS. 5A-5B). The internal structure 738 may include openings to house the magnets 740. The openings may accept surfaces of the movable components 736. In some examples, the internal structure 738 may include at least two components. Each of the two components may be shaped to form two “E”s facing one another.

The magnets 740 may provide magnetic force to the components of the exciter 725. In some cases, the magnets 740 may produce a magnetic force in response to an electric field generated by the wire coil 742. The magnets 740 may produce a magnetic force to adjust a position of the movable component 736 to cause the suspension spring 732 to extend and contract according to the electric field generated by the wire coil 742. For example, the wire coil 742 may generate an electric field corresponding to an signal to the sensor, such that the magnets 740 produces a magnetic force in response to the electric field. The magnetic force may cause movement of the movable component 736 (such as an oscillation). For example, the magnets 740 may include a first magnet with a north-south configuration and a second magnet with a south-north configuration, such that the placement of the magnets in the exciter 750 repel each other. In this way, when the wire coil 742 generates an electric field, the magnets 740 may each respectively generate a magnetic field with a force corresponding to the electric field from the wire coil 742. Accordingly, the magnetic fields may repel one another with varying strength, causing the movable component 736 to adjust a position corresponding to the strength of the magnetic fields. The movement of the movable component 736 may cause the exciter nob 728 to oscillate which may cause the exciter nob 728 to strike a patient. The exciter nob 728 striking the patient may result in an acoustic signal to travel within the patient that corresponds to the signal, and accordingly, received by the sensor. The movement of the movable component 736 provides movement to the mass 730 allowing the exciter 725 to obtain a signal that corresponds to the movement of the exciter nob 728. In some cases, the magnets 740 may include at least two magnets. The magnets 740 may have predetermined dimensions (for example, the outer diameter, inner diameter, and thickness disclosed for the magnet 504 in FIGS. 5A-5B). The magnets 740 may be constructed of various materials (for example, the materials disclosed for the magnet 504 in FIGS. 5A-5B). In some cases, the magnets 740 is in a permanent (or non-permanent) position in a housing of the exciter 725. In some cases, the exciter 725 may include the magnets 740 as part of a voice coil actuator (including a moving coil actuator or a moving magnet actuator) and/or a solenoid.

The wire coil 742 may generate an electric field in response to receiving an signal. The wire coil 742 may receive the signal as an analog (or digital) electrical signal corresponding to a range of frequencies as described herein (for example, the range of frequencies disclosed for the wire coil 508 in FIGS. 5A-5B). The wire coil 742 may be constructed of various materials as described herein (for example, the materials disclosed for the wire coil 508 in FIGS. 5A-5B). The wire coil 742 may be constructed of predetermined dimensions as described herein (for example, the dimensions disclosed for the wire coil 508 in FIGS. 5A-5B). The wire coil 742 may include a material thickness as described herein (for example, the material thickness disclosed for the wire coil 508 in FIGS. 5A-5B). The wire coil 742 may include an outer diameter, an inner diameter, and a thickness as described herein (for example, the outer diameter, inner diameter, and thickness disclosed for the wire coil 508 in FIGS. 5A-5B). In some cases, the outer diameter of the wire coil 742 may be lesser (or greater) than a width of the movable component 736. For example, the wire coil 742 may be wrapped around a central region of the movable component 736 that is narrower than the widest portion of the movable component. In some cases, the wire coil 742 is in a permanent (or non-permanent) position in a housing. In some cases, the exciter 725 may include the wire coil 742 as part of a voice coil actuator (including a moving coil actuator or a moving magnet actuator) and/or a solenoid.

FIGS. 7C-7D illustrate magnetic characteristic graphs 750 for the exciter. As illustrated in FIGS. 7C-7D, the graphs 750 include a diagrammatic version of the movable component 751, first regions of an increased magnetic field 752A,B, an axis 754, and second regions of an increased magnetic field 756A,B. As illustrated in FIG. 7C, the movable component 751 may be in a first position. The first position may correspond to an signal driving a wire coil to generate an electric field and causing magnets to produce a magnetic force. The magnetic force may adjust the movable component 751 to be in the first position. The first position may be a maximum moment to position an exciter nob farthest downward along the axis 754. The first regions of an increased magnetic field 752A,B illustrate an interaction between the magnets and the movement of the movable component 751 in the first position. As illustrated in FIG. 7D, the movable component 751 may be a second position. Similar to the first position, the second position may correspond to an signal driving a wire coil to generate an electric field and causing magnets to produce a magnetic force. The magnetic force may adjust the movable component 751 to be in the second position. The second position may be a minimum moment to position the exciter nob farthest upward along the axis 754. The second regions of an increased magnetic field 756A,B illustrate an interaction between the magnets and the movement of the movable component 751 in the second position.

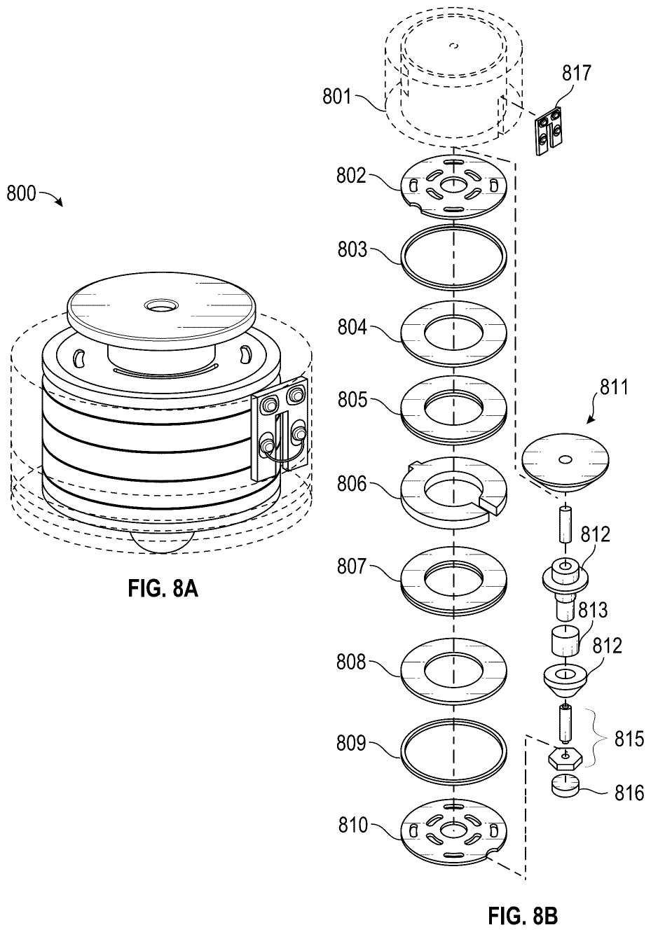

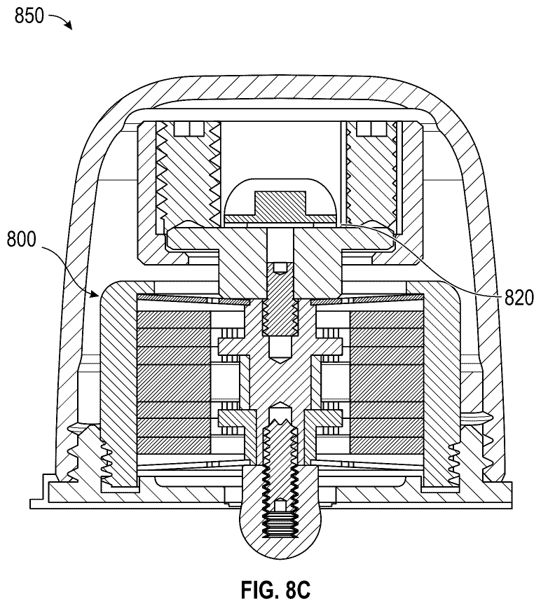

FIG. 8A-8B illustrate various views of an exciter (may also be referred to herein as a “magnetic exciter”). FIG. 8A illustrates a perspective view of the exciter. FIG. 8B illustrates a perspective view of an example exploded view of the exciter 800. As illustrated in FIG. 8B, the exciter 800 may include a housing 801, spring 802, spacer 803, washer 804, magnet 805, washer 806, magnet 807, washer 808, spacer 809, spring 810, a mass 811, a bobbin 812, a wire coil 813, a plunger 815, an exciter nob 816, and a wire connector 817. The exciter 800 may receive a signal from a signal generation source. In some cases, the signal generation source may be local to the sensor (or external signal generation source coupled to the sensor). The signal generation source may transmit the signal from an exciter driver (such as exciter driver 910 in FIG. 9) to an RD connector of the sensor (such as connector 416, 466 in FIGS. 4A and 4B). The RD connector may connect and transmit the signal to the wire coil 813. The signal may be an analog (or digital) electrical signal corresponding to a range of frequencies. The wire coil 813 may generate an electric field according to the signal. In some cases, the electric field generated by the wire coil 813 may oscillate according to oscillations of the frequencies of the signal. In some examples, the exciter may push an exciter nob, for example, according to a signal. The exciter 800 may include any component, material, feature, capability, function as described with respect to any other exciter herein (such as, exciter 500, 600, and 700).

In some examples, the mass 811 may attach to the bobbin 812, which may attach to the exciter nob 816. In some cases, the wire coil 813 wraps around the bobbin 812. The bobbin 812 may adjust position of the exciter nob 816 and the mass 811. The bobbin 812 may attach to the wire coil 813, such that the wire coil 813 is wrapped around part of the bobbin 812. The attachment of the bobbin 812 and the wire coil 813 may cause movement of the bobbin 812 and the wire coil 813 in response to changes in an electric field generated by the wire coil 813 (and corresponding magnetic force by the magnets 805, 807). In some cases, the magnets 805, 807 may produce a magnetic force in response to an electric field generated by the wire coil 813. The magnets 805, 807 may produce a magnetic force to oscillate the bobbin 812 and the wire coil 813, which may cause the suspension springs 802, 810 to extend and contract. For example, the wire coil 813 may generate an electric field corresponding to an applied signal to the sensor, such that the magnets 805, 807 produce a magnetic force in response to the electric field. The magnetic force may cause movement of the bobbin 812 and the wire coil 813 to cause the suspension spring 802, 810 to extend and contract (such as an oscillation). In some instances, the movement of the suspension spring 802, 810 may cause the exciter nob 816 and a mass 811 to oscillate which may cause the exciter nob 816 to strike a patient and, accordingly, the sensor may obtain a signal corresponding to the exciter nob 816 oscillating.

The housing 801 may provide structural support to the exciter components. In some cases, the housing 801 may provide an opening for the exciter nob 816. The housing 801 may be positioned to contact the patient. An opening of the housing 801 may be wide enough to give clearance to the exciter nob 816 as the suspension springs 802, 810 flex. The housing 801 may be constructed of various materials (for example, the materials disclosed for the housing 702 in FIG. 7A).

The exciter nob 816 may adjust a position in response to extension and contraction of the suspension springs 802, 810. The exciter nob 816 may attach to the suspension spring 810 via a fastener. The exciter nob 816 may attach to the bobbin 812 and the suspension spring 810, such that the bobbin 812 and the suspension spring 810 form a base for the exciter nob 816. The exciter nob 816 may be constructed with various materials (for example, the materials disclosed for the exciter nob 554 in FIG. 5B).

The mass 811 may adjust a position in response to extension and contraction of the suspension spring 802. The mass 811 may obtain a signal corresponding to the movement of the exciter nob 816, such as a secondary signal to replicate movement of the exciter nob 816. In this manner, the signal receiver 730 may provide the sensor (for example, to a feedback detector 404,454 in FIGS. 4A-4B) with the signal for computation of a patient's physiological parameters (such as measuring blood pressure). The mass 811 may include bio-compatible materials. In some cases, the mass 811 may be metal and/or a metallic alloy. In some cases, the mass 811 may be magnetic or non-magnetic metal (or metallic alloy). For example, the mass 811 may be stainless steel, aluminum, copper, titanium, cobalt chrome, magnesium, gold, platinum, silver, iridium, nitinol, and/or tantalum, and/or any other metal (or alloy) suitable for the applications disclosed herein. In some cases, the stainless steel may include 200 series stainless steel, 300 series stainless steel, 400 series stainless steel, duplex steel, 17-4PH, and/or carbon steel. In some cases, the mass 811 may be rubber material. For example, the mass 811 may be silicone rubber (liquid silicone rubber and/or high consistency rubber). In some cases, the mass 811 may be plastic. For example, the mass 811 may be polyethylene, polypropylene, PMMA, PVC, polyamide, ABS, polycarbonate, or any other plastic suitable for the applications disclosed herein.

The suspension spring 802 may oscillate in response to an applied signal. The suspension spring 802 may adjust position according to a state of the wire coil 813. When the wire coil 813 is in a non-driven state, the suspension spring 802 may apply mechanical force to the exciter nob 816 and the mass 811. The suspension spring 802 may pull the exciter nob 816 towards the internal structure 738 such that the exciter nob 816 is mechanically stopped from moving towards the internal structure 738 by the bobbin 812. When the wire coil 813 is in a driven state, the suspension spring 802 may extend and compress such that the bobbin 812 oscillates from a minimum position to a maximum position. In some cases, the minimum position may be when the mass 811 is closest to the housing 801 along an axis aligned with motion of the exciter nob 816. In some cases, the maximum position may be when the mass 811 is farthest from the housing 801 along an axis aligned with motion of the exciter nob 816. The range between the minimum position and the maximum position may correspond to an amplitude of a signal, which may cause the wire coil 813 to generate an electric field and the magnets 805, 807 to produce magnetic force. In some cases, the suspension spring 802 may be a compression spring, a tension spring, constant force spring, spring-powered reel, or a torsion spring. In some cases, the suspension spring 802 may be constructed of various materials (for example, the materials disclosed for the suspension spring 552 in FIG. 5B). In some cases, the suspension spring 802 may include predetermined dimensions (for example, the layers, turns, material thickness, outer diameter, inner diameter, and thickness disclosed for the suspension spring 552 in FIG. 5B).

The bobbin 812 may adjust a position in response to changes in an electric field generated by the wire coil 813 (and corresponding magnetic force by the magnets 805, 807). The bobbin 812 may be constructed of various materials as described herein (for example, the materials disclosed for the back plate 502 and the top plate 506 in FIGS. 5A-5B). The bobbin 812 may include rectangular parallel surfaces crossed with a rectangular perpendicular surface connecting the parallel surfaces. In some examples, the bobbin 812 forms two plus signs stacked on top of each other. Ends of the parallel surfaces may be adjacent to the magnets 805, 807. Both ends of the perpendicular surface may be attached to the suspension spring 802, 810. In some cases, one end of the perpendicular surface may be attached to the exciter nob 816, the signal receiver 730, and the fasteners 734A,B. In this manner, the perpendicular surface may mechanically drive the exciter nob 816 and provide the same movement for the signal receiver 730.

The spacers 803, 809 and the washers 804, 806, 808 may provide separation at least between the suspension spring 802, 810 and the magnets 805, 807. The spacers 803, 809 and the washers 804, 806, 808 may be constructed of various materials as described herein (for example, the materials disclosed for the back plate 502 and the top plate 506 in FIGS. 5A-5B).

The magnets 805, 807 may provide magnetic force to the components of the exciter 725. In some cases, the magnets 805, 807 may produce a magnetic force in response to an electric field generated by the wire coil 813. The magnets 805, 807 may produce a magnetic force to adjust a position of the bobbin 812 to cause the suspension spring 802, 810 to extend and contract according to the electric field generated by the wire coil 813. For example, the wire coil 813 may generate an electric field corresponding to an signal to the sensor, such that the magnets 805, 807 produces a magnetic force in response to the electric field. The magnetic force may cause movement of the bobbin 812 (such as an oscillation). The movement of the bobbin 812 may cause the exciter nob 816 to oscillate which may cause the exciter nob 816 to strike a patient. The exciter nob 816 striking the patient may result in an acoustic signal to travel within the patient that corresponds to the signal to the sensor. The movement of the bobbin 812 provides movement to the signal receiver 730 allowing the exciter 725 to obtain a signal that corresponds to the movement of the exciter nob 816. In some cases, the magnets 805, 807 may include at least two magnets. The magnets 805, 807 may have predetermined dimensions (for example, the outer diameter, inner diameter, and thickness disclosed for the magnet 504 in FIGS. 5A-5B). The magnets 805, 807 may be constructed of various materials (for example, the materials disclosed for the magnet 504 in FIGS. 5A-5B). In some cases, the magnets 805, 807 is in a permanent (or non-permanent) position in a housing of the exciter 725. In some cases, the exciter 725 may include the magnets 805, 807 as part of a voice coil actuator (including a moving coil actuator or a moving magnet actuator) and/or a solenoid.

The wire coil 813 may generate an electric field in response to receiving an signal. The wire coil 813 may receive the signal as an analog (or digital) electrical signal corresponding to a range of frequencies as described herein (for example, the range of frequencies disclosed for the wire coil 508 in FIGS. 5A-5B). The wire coil 813 may be constructed of various materials as described herein (for example, the materials disclosed for the wire coil 508 in FIGS. 5A-5B). The wire coil 813 may be constructed of predetermined dimensions as described herein (for example, the dimensions disclosed for the wire coil 508 in FIGS. 5A-5B). The wire coil 813 may include a material thickness as described herein (for example, the material thickness disclosed for the wire coil 508 in FIGS. 5A-5B). The wire coil 813 may include an outer diameter, an inner diameter, and a thickness as described herein (for example, the outer diameter, inner diameter, and thickness disclosed for the wire coil 508 in FIGS. 5A-5B). In some cases, the outer diameter of the wire coil 813 may be lesser (or greater) than a width of the bobbin 812. For example, the wire coil 813 may be wrapped around a central region of the bobbin 812 that is narrower than the widest portion of the bobbin 812. In some cases, the wire coil 813 is in a permanent (or non-permanent) position in a housing. In some cases, the exciter 800 may include the wire coil 813 as part of a voice coil actuator (including a moving coil actuator or a moving magnet actuator) and/or a solenoid.

The wire connector 817 may provide a signal to the wire coil 813 to generate the electric field. In some cases, the wire connector 817 may attach to the sensor (such as, sensor 400, 450 in FIGS. 4A and 4B, as disclosed herein). In this way, the wire connector may provide the signal to the wire coil 813 and the sensor. By providing the signal to both the wire coil 813 and the sensor, the sensor may obtain the signal without interference from the body of the patient. The signal provided to the sensor may be a reference signal for assessing a blood pressure of the patient.

FIG. 8C illustrates a cross-sectional view of the exciter 800 and housing components 850. The exciter 800 may be housed within the housing component 850. In some examples, the exciter 800 may attach to a sensor at sensor portion 820. For example, the sensor may be placed on top of the mass 811, such that the wire connector 817 may attach to the sensor.

FIG. 9 illustrates an architecture 900 for an example embodiment of a blood pressure monitoring system. The architecture 900 depicted in FIG. 9 includes an arrangement of computer hardware and software modules that may be used to implement aspects of the present disclosure. The hardware modules may be implemented with physical electronic devices, as discussed in greater detail below. The architecture 900 may include more (or fewer) elements than those shown in FIG. 9. It is not necessary, however, that all of these generally conventional elements be shown in order to provide an enabling disclosure. Additionally, the architecture 900 illustrated in FIG. 9 may be used to implement one or more of the other components as disclosed herein. As illustrated, the general architecture may include a system 902, an exciter device 920, an interface 830, and an external device 840. The system 902 may include a controller 904, a power supply 906, a communication driver 908, an exciter driver 910, a DAC 912, an ADC 913, sensor(s) 914, and a system interface 915.

In some instances, the system 902 may include a printed circuit board (PCB) including conductive traces to interconnect the components of the system 902. In some cases, the system 902 may be coupled to the exciter device 920, the external interface 830, and/or the external device 840. The system 902 may communicate with the exciter device 920 to control operation of the exciter device 920. For example, the system 902 may instruct the exciter driver 910 to communicate with the exciter device 920 to begin emitting a signal into the patient. In some cases, the system 902 may instruct the exciter device 920 to transmit detected signal data to the system 902 for computing the patient's blood pressure.

In some instances, the controller 904 may compute the blood pressure of the patient in part by determining a system response between the exciter and the detector of the exciter device 920 (such as by computing the equation 312 in FIG. 3). The system 902 may be used to compute other physiological parameters of the patient. For example, measuring parameters and computing a cardiac output. In some cases, the controller 904 may be coupled to the exciter device 920 to interface with sensors on the exciter device 920. In some cases, the controller 904 may be connected to the exciter device 920 via wired or wireless communication methods (for example, RD connector, BLE, WiFi, NFC, or another communication protocol appropriate for the desired application). In some cases, the controller 904 may provide communication between the components of the system 902, the exciter device 920, the external interface 830, and the external device 840. In some instances, the controller 904 may include an Ambiq® Apollo4 Blue system on chip (SOC) (which may include an I2C and/or pulse-density modulation (PDM) interface).

In some instances, the power supply 906 may provide power to the system 902. The power supply 906 may include power supply circuits, such as a battery and/or a pluggable power line.

In some instances, the communication driver 908 may allow integration of various communication protocols used by the components internal of the system 902 to interface with the other components and/or with the exciter device 920, the external interface 830, and the external device 840. In some cases, the communication driver 908 may include a communication bus to interface the system 902 components as a central hub. In some cases, the communication driver 908 may include a signal adaptor. The signal adaptor may transform a first signal to a second signal. For example, the signal adapter may transform a signal from an RD connector to a common protocol format to interface with the components of the system 902. In some cases, the communication driver 908 may interface various communication protocols such that the components of the system 902 and other components may properly communicate (for example, RD connector, BLE, WiFi, NFC, or another communication protocol appropriate for the desired application).

In some instances, the exciter driver 910 may control the exciter of the exciter device 920. For example, the exciter driver 910 may instruct the exciter of the exciter device 920 to emit a signal at a range of frequencies. In some cases, the exciter driver 910 may be connected to the exciter device 920 via wired or wireless communication methods (for example, RD connector, BLE, WiFi, NFC, or another communication protocol appropriate for the desired application). The signal may include instructions for the exciter device 920 to transmit at least one of an acoustic signal, an electrical signal, a magnetic signal, RF signal, and/or mechanical signal, or another signal appropriate for measuring blood pressure. The exciter driver 910 may provide analog and/or digital signals to the exciter device 920.

In some instances, the DAC 912 may include DAC hardware. In some cases, the DAC may be coupled to an analog out port. The analog out port may connect the DAC with the external device 840. In some cases, the DAC 912 may be connected to the external device 840 via wired or wireless communication methods (for example, RD connector, BLE, WiFi, NFC, or another communication protocol appropriate for the desired application). In some instances, the ADC 913 may include ADC hardware. In some cases, the ADC may be coupled to a hydrostatic sensor of the sensor(s) 914. In some instances, the sensor(s) 914 may include the hydrostatic sensor. The hydrostatic sensor may provide a heart-level reference for blood pressure measurements. For example, the hydrostatic sensor may include an accelerometer configured to measure a body position of at least part of the patient. In some cases, the hydrostatic sensor may detect an arm position of the patient. The sensor(s) 914 may be coupled to a hydrostatic line of the exciter device 920. In some cases, the exciter device 920 is positioned on an arm of the patient. In this manner, when the patient moves their arm, the hydrostatic line of the exciter device 920 may indicate to the system 902 that the patient's body position changed. In some instances, the system interface 915 may include a 1-Wire interface. The system interface 915 may be coupled to a memory device of the exciter device 920.

In some instances, the exciter device 920 may include an exciter, a detector, an inertial measurement unit (IMU), a memory device, a hydrostatic line, and an RD connector. The exciter device 920 may include the sensors as described herein (such as sensors 400, 450 in FIGS. 4A and 4B). In some instances, the external interface 830 may include a medical device interface. For example, the medical device interface may include an MOC-9® interface. In some instances, the external device 840 may include medical devices used for measuring hemodynamic parameters for the patient. For example, the data provided by the exciter device 920 may be used for determining hemodynamic parameters, such as mean arterial pressure (MAP), stroke volume (SV), cardiac output (CO), cardiac index (CI), system vascular resistance (SVR), oxygen delivery (DO₂), stroke volume variation (SVV), heart rate (HR), changes in pressure over time (dPdt), dynamic arterial elastance (EA-dyn), and pulse pressure variation (PPV). In some examples, the medical devices may include a Masimo LiDCO™ hemodynamic monitoring platform.

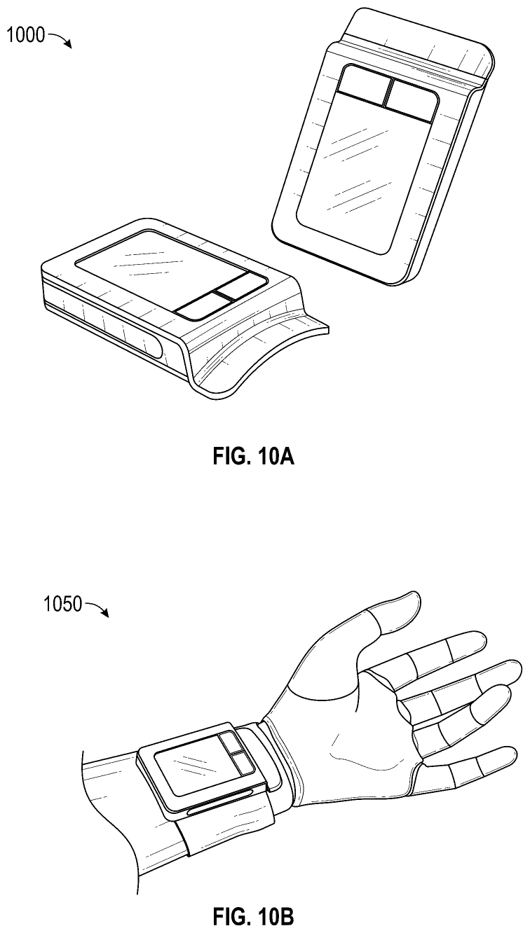

FIGS. 10A-10B illustrate an example embodiment of a blood pressure monitoring system with a built-in display. The illustrated embodiment can include all components of the blood pressure monitoring system (for example, waveform/signal generator, processor, battery, etc.) in a single wearable package. The display can be used, for example, to output measurements to the user in graphical or text format. Though not seen, the exciter and detector(s) can be provided in the underside of the unit so as to be in contact with the patient's skin when worn. As shown in FIG. 10A, a blood pressure monitoring system 1000 can also include an indicator to show how the unit should be positioned with respect to the patient's artery, for example, the radial artery or the ulnar artery. FIG. 10B shows a blood pressure monitoring system 1050 attached to the patient's forearm with a wrist strap and worn like a wristwatch.

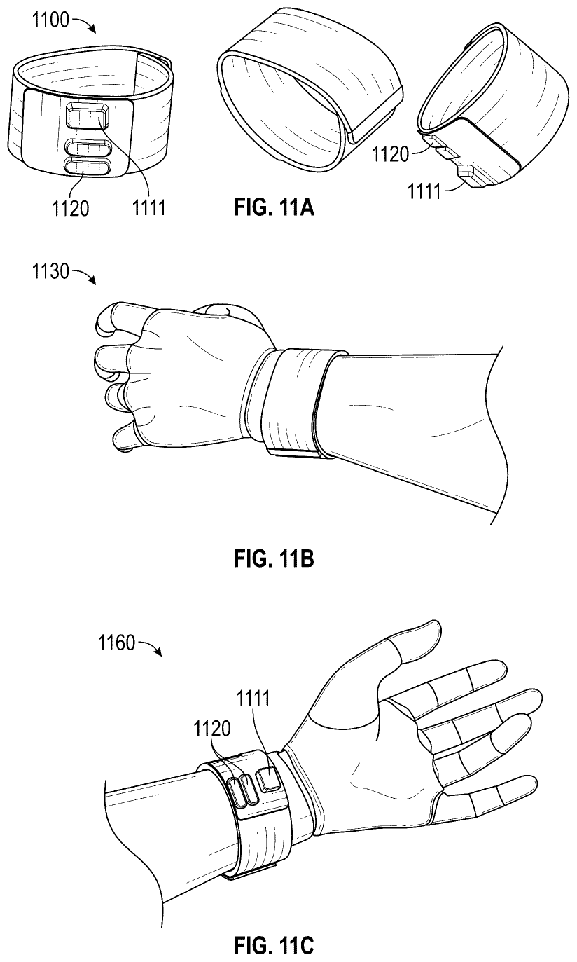

FIGS. 11A-11C illustrate an example embodiment of a blood pressure monitoring system 1100, 1130, 1160 with an exciter 1110 and detectors 1120 integrated in a wristband. The wristband can include a controller to provide a signal input to the exciter 1110 and to capture output signals from the detectors 1120. The wristband can also include a wireless communication module to provide data to an external device, such as a smartphone, to process the output signals and display the blood pressure measurements. The wristband can include windows through the wristband material to allow the exciter 1110 and the detectors 1120 to be in contact with the patient's skin when the wristband is worn.

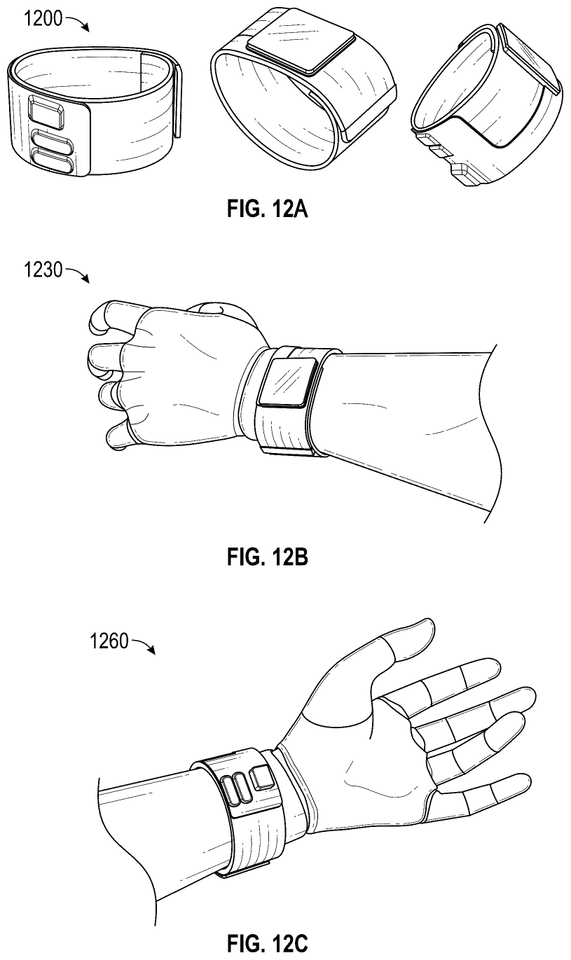

FIGS. 12A-12C illustrate an example embodiment of a blood pressure monitoring system 1200, 1230, 1260 which is similar to that of FIGS. 11A-11C, but which additionally includes a built-in display.

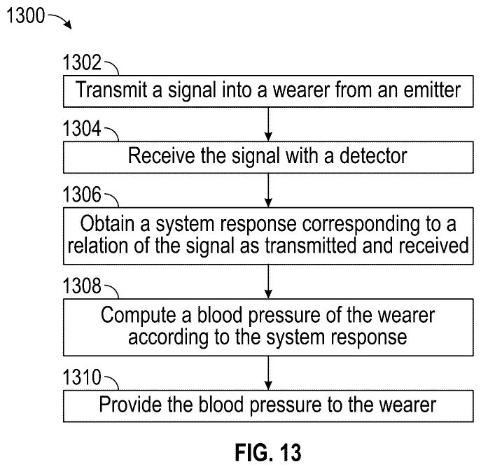

FIG. 13 is one illustrative routine 1300 for operating a sensor as disclosed herein. The routine 1300 may be carried out, for example, by the architecture 900 of FIG. 9. The routine 1300 begins at block 1302, where the architecture may transmit a signal into a wearer from an emitter. In some instances, the system may transmit the signal into the wearer with a sensor (such as sensor 400,450 of FIGS. 4A-4B). The sensor may emit a signal into the wearer. For example, the signal may include at least one of an acoustic signal, an electrical signal, a magnetic signal, RF signal, and/or mechanical signal, or another signal appropriate for measuring blood pressure.

At block 1304, the architecture may receive the signal with a detector. The system may obtain a received signal with the detector. For example, the detector may include a plurality of detectors (such as the sensors 400, 450 of FIGS. 4A-4B).

At block 1306, the architecture may obtain a system response corresponding to a relation of the signal as transmitted and received. In some instances, the system response may include a combination of the emitted signal and the detected signal (for example, according to equation 312 in FIG. 3).

At block 1308, the architecture may compute a blood pressure of the wearer according to the system response. In some instances, the architecture may compute the blood pressure by applying algorithms to process the emitted signal and the detected signal.

At block 1310, the architecture may provide the blood pressure to the wearer. In some instances, the architecture may display the blood pressure measurement to the wearer using a display as part of the patient monitoring platform (such as patient monitoring platform 112 in FIG. 1).

### 1. Additional Embodiments and Terminology

Some inventive aspects of the disclosure are set forth in the following clauses:

Clause 1. A blood pressure monitoring system, comprising: an exciter configured to produce an acoustic signal, the exciter being provided on a first substrate portion; a detector spaced apart from the exciter, the detector being configured to detect the acoustic signal and to produce an electrical output signal, the detector being provided on a second substrate portion that is mechanically or acoustically decoupled from the first substrate portion, wherein the detector comprises at least four detectors; and a processor configured to determine a blood pressure measurement from the electrical output signal.

Clause 2. The blood pressure monitoring system of clause 1, wherein the at least four detectors are separated by at least one gap.

Clause 3. The blood pressure monitoring system of any of the preceding clauses, wherein the detector comprises five detectors.

Clause 4. The blood pressure monitoring system of any of the preceding clauses, further comprising a memory device.

Clause 5. The blood pressure monitoring system of any of the preceding clauses, further comprising an inertial measurement unit (IMU).

Clause 6. The blood pressure monitoring system of any of the preceding clauses, wherein the first substrate portion and the second substrate portion are separated by a gap.

Clause 7. The blood pressure monitoring system of any of the preceding clauses, wherein the first substrate portion and the second substrate portion are separated by acoustically absorptive material.

Clause 8. The blood pressure monitoring system of any of the preceding clauses, wherein the processor is configured to determine the blood pressure measurement based on a measured phase delay between an electrical input signal and the electrical output signal.

Clause 9. The blood pressure monitoring system of any of the preceding clauses, wherein the first substrate portion and the second substrate portion are flexible.

Clause 10. The blood pressure monitoring system of any of the preceding clauses, further comprising a flexible circuit that connects the exciter and the detector to an electrical connector.

Clause 11. The blood pressure monitoring system of any of the preceding clauses, wherein the exciter and the detector comprise a piezo device or a microelectromechanical system.

Clause 12. The blood pressure monitoring system of any of the preceding clauses, further comprising an attachment element configured to attach the exciter and the detector to a forearm of a patient over a radial artery.

Clause 13. The blood pressure monitoring system of any of the preceding clauses, wherein the attachment element comprises an adhesive substrate.

Clause 14. The blood pressure monitoring system of any of the preceding clauses, further comprising an alignment indicator to align a measurement axis of the exciter and the detector to the radial artery.

Clause 15. The blood pressure monitoring system of any of the preceding clauses, further comprising an attachment element configured to attach the exciter and the detector to a palm side of a wrist on a forearm of a patient.

Clause 16. The blood pressure monitoring system of any of the preceding clauses, further comprising an attachment element configured to attach the exciter and the detector to a patient, wherein the exciter is closer to a hand of the patient than the detector.

Clause 17. The blood pressure monitoring system of any of the preceding clauses, further comprising an attachment element configured to attach the exciter and the detector to a patient, wherein the detector is closer to a hand of the patient than the exciter.

Clause 18. The blood pressure monitoring system of any of the preceding clauses, further comprising an attachment element configured to attach the exciter and the detector to a patient, wherein the exciter and the detector are attached parallel to a length of an arm of the patient from an elbow to a wrist of the patient.

Clause 19. The blood pressure monitoring system of any of the preceding clauses, further comprising an attachment element configured to attach the exciter and the detector to a patient, wherein the exciter is positioned over a radial artery of the patient.

Clause 20. The blood pressure monitoring system of any of the preceding clauses, further comprising an attachment element configured to attach the exciter and the detector to a patient, wherein the detector is positioned over a radial artery of the patient.

Clause 21. A blood pressure monitoring system, comprising: an exciter configured to produce an acoustic signal, the exciter being provided on a substrate; a detector spaced apart from the exciter, the detector being configured to detect the acoustic signal and to produce an electrical output signal, the detector being provided on the substrate, wherein the detector comprises at least four detectors; and a processor configured to determine a blood pressure measurement from the electrical output signal, wherein a gap in the substrate is along a line path from the exciter to the detector.

Clause 22. The blood pressure monitoring system of any of the preceding clauses, wherein the line path is in a straight line centered on the exciter from the exciter to the detector.

Clause 23. The blood pressure monitoring system of any of the preceding clauses, wherein the line path is off center from the exciter to the detector.

Clause 24. The blood pressure monitoring system of any of the preceding clauses, further comprising a memory device.

Clause 25. The blood pressure monitoring system of any of the preceding clauses, further comprising an inertial measurement unit (IMU).

Clause 26. The blood pressure monitoring system of any of the preceding clauses, wherein a path from the exciter to the detector via the substrate is longer than the line path from the exciter to the detector.

Clause 27. The blood pressure monitoring system of any of the preceding clauses, wherein the path from the exciter to the detector via the substrate is at least two times longer than a distance between the exciter and the detector.

Clause 28. The blood pressure monitoring system of any of the preceding clauses, wherein the path from the exciter to the detector via the substrate is at least five times longer than a distance between the exciter and the detector.

Clause 29. The blood pressure monitoring system of any of the preceding clauses, wherein the path from the exciter to the detector via the substrate comprises acoustically absorptive material.

Clause 30. The blood pressure monitoring system of any of the preceding clauses, wherein the processor is configured to determine the blood pressure measurement based on a measured phase delay between an electrical input signal and the electrical output signal.

Clause 31. The blood pressure monitoring system of any of the preceding clauses, wherein the exciter and the detector comprise a piezo device or a microelectromechanical system.

Clause 32. The blood pressure monitoring system of any of the preceding clauses, further comprising an attachment element configured to attach the exciter and the detector to a forearm of a patient over a radial artery.

Clause 33. The blood pressure monitoring system of any of the preceding clauses, wherein the attachment element comprises an adhesive substrate.

Clause 34. The blood pressure monitoring system of any of the preceding clauses, further comprising an alignment indicator to align a measurement axis of the exciter and the detector to the radial artery.

Clause 35. The blood pressure monitoring system of any of the preceding clauses, further comprising an attachment element configured to attach the exciter and the detector to a palm side of a wrist on a forearm of a patient.

Clause 36. The blood pressure monitoring system of any of the preceding clauses, further comprising an attachment element configured to attach the exciter and the detector to a patient, wherein the exciter is closer to a hand of the patient than the detector.

Clause 37. The blood pressure monitoring system of any of the preceding clauses, further comprising an attachment element configured to attach the exciter and the detector to a patient, wherein the detector is closer to a hand of the patient than the exciter.

Clause 38. The blood pressure monitoring system of any of the preceding clauses, further comprising an attachment element configured to attach the exciter and the detector to a patient, wherein the exciter and the detector are attached parallel to a length of an arm of the patient from an elbow to a wrist of the patient.

Clause 39. The blood pressure monitoring system of any of the preceding clauses, further comprising an attachment element configured to attach the exciter and the detector to a patient, wherein the exciter is positioned over a radial artery of the patient.

Clause 40. The blood pressure monitoring system of any of the preceding clauses, further comprising an attachment element configured to attach the exciter and the detector to a patient, wherein the detector is positioned over a radial artery of the patient.

Clause 41. A blood pressure monitoring system comprising: an exciter configured to produce an acoustic signal; a plurality of detectors spaced apart from the exciter, the plurality of detectors being configured to detect the acoustic signal and to produce a plurality of electrical output signals, wherein the plurality of detectors comprises at least four detectors; and a processor configured to determine a blood pressure measurement from the plurality of electrical output signals.

Clause 42. The blood pressure monitoring system of any of the preceding clauses, wherein the plurality of detectors are separated by at least one gap.

Clause 43. The blood pressure monitoring system of any of the preceding clauses, wherein the plurality of detectors are mechanically decoupled from the exciter.

Clause 44. The blood pressure monitoring system of any of the preceding clauses, wherein the plurality of detectors are mechanically decoupled from one another.

Clause 45. The blood pressure monitoring system of any of the preceding clauses, wherein the plurality of detectors are arranged in a linear array.

Clause 46. The blood pressure monitoring system of any of the preceding clauses, wherein the plurality of detectors are arranged in a two-by-two array.

Clause 47. The blood pressure monitoring system of any of the preceding clauses, wherein, using the plurality of electrical output signals, the processor is further configured to determine an arterial propagation distance traveled by the acoustic signal and to compute the blood pressure measurement using the arterial propagation distance.

Clause 48. The blood pressure monitoring system of any of the preceding clauses, wherein a distance between an exciter and a detector corresponds to each of the plurality of detectors, and wherein the processor is further configured to determine whether the distance corresponds to the arterial propagation distance.

Clause 49. The blood pressure monitoring system of any of the preceding clauses, wherein the processor is further configured to determine the blood pressure measurement using the arterial propagation distance and a measured phase delay between an electrical input signal and one or more electrical output signals.

Clause 50. The blood pressure monitoring system of any of the preceding clauses, wherein one or more of the plurality of detectors is mechanically decoupled from the exciter and wherein one or more of the plurality of detectors is not mechanically decoupled from the exciter.

Clause 51. The blood pressure monitoring system of any of the preceding clauses, wherein the processor is further configured to use the one or more electrical output signals corresponding to the plurality of detectors which is not mechanically decoupled from the exciter to determine one or more characteristics of the acoustic signal.

Clause 52. The blood pressure monitoring system of any of the preceding clauses, wherein the processor is further configured to adjust the plurality of electrical output signals or the blood pressure measurement using the one or more characteristics of the acoustic signal.

Clause 53. The blood pressure monitoring system of any of the preceding clauses, wherein the blood pressure measurement comprises systolic pressure, diastolic pressure, mean arterial pressure, or instantaneous arterial pressure.

Clause 54. The blood pressure monitoring system of any of the preceding clauses, wherein the electrical input signal comprises a plurality of frequencies.

Clause 55. The blood pressure monitoring system of any of the preceding clauses, further comprising a memory device.

Clause 56. The blood pressure monitoring system of any of the preceding clauses, further comprising an inertial measurement unit (IMU).

Clause 57. The blood pressure monitoring system of any of the preceding clauses, further comprising an attachment element configured to attach the exciter and the plurality of detectors to a palm side of a wrist on a forearm of a patient.

Clause 58. The blood pressure monitoring system of any of the preceding clauses, further comprising an attachment element configured to attach the exciter and the plurality of detectors to a patient, wherein the exciter is closer to a hand of the patient than the plurality of detectors.

Clause 59. The blood pressure monitoring system of any of the preceding clauses, further comprising an attachment element configured to attach the exciter and the plurality of detectors to a patient, wherein the plurality of detectors are closer to a hand of the patient than the exciter.

Clause 60. The blood pressure monitoring system of any of the preceding clauses, further comprising an attachment element configured to attach the exciter and the plurality of detectors to a patient, wherein the exciter and the plurality of detectors are attached parallel to a length of an arm of the patient from an elbow to a wrist of the patient.

Clause 61. The blood pressure monitoring system of any of the preceding clauses, further comprising an attachment element configured to attach the exciter and the plurality of detectors to a patient, wherein the exciter is positioned over a radial artery of the patient.

Clause 62. The blood pressure monitoring system of any of the preceding clauses, further comprising an attachment element configured to attach the exciter and the plurality of detectors to a patient, wherein the plurality of detectors are positioned over a radial artery of the patient.

Clause 63. An exciter, comprising: a housing configured to provide structural support for the exciter, wherein the housing comprises an opening; an exciter nob coupled to the housing and configured to adjust a position via the opening of the housing; a suspension spring coupled to the exciter nob and the housing, wherein the suspension spring extends and retracts in response to a driven force; an internal structure coupled to the suspension spring, wherein the internal structure is configured to provide structural support for the suspension spring; a movable component coupled to the suspension spring and the exciter nob, wherein the movable component is configured to adjust a position in response to the driven force, wherein the position of the movable component adjusts the position of the exciter nob; at least one magnet coupled to the internal structure, wherein the at least one magnet is configured to produce at least in part the driven force, wherein the driven force comprises magnetic force; and a wire coil coupled to the movable component, wherein the wire coil is configured to receive an applied signal and generate an electric field in response to the applied signal, wherein the electric field interacts with the at least one magnet to drive the magnetic force.

Clause 64. The exciter of clause 63, further comprising a signal receiver coupled to the suspension spring and the movable component, wherein the signal receiver is configured to obtain a secondary signal to replicate movement of the exciter nob.

Clause 65. A blood pressure monitoring system comprising: an exciter configured to produce an acoustic signal; a plurality of detectors spaced apart from the exciter, the plurality of detectors being configured to detect the acoustic signal and to produce a plurality of electrical output signals, wherein the plurality of detectors are mechanically or acoustically decoupled from one another; and a processor configured to determine a blood pressure measurement from the plurality of electrical output signals.

Clause 66. A blood pressure monitoring system comprising: an exciter configured to transmit a signal into tissue of a patient; and a detector configured to receive the signal that is reflected from an artery of the patient, wherein the blood pressure monitoring system is configured to detect changes in blood pressure.

Clause 67. The blood pressure monitoring system of any of the preceding clauses, used with any of clauses 1-66 or any other clauses or claims herein.

Clause 68. The blood pressure monitoring system of any of the preceding clauses, wherein the changes in blood pressure are detected according to a system response based on the transmitted signal and the received signal.

Clause 69. The blood pressure monitoring system of any of the preceding clauses, wherein the exciter is coupled to a first substrate and the detector is coupled to a second substrate.

Clause 70. The blood pressure monitoring system of any of the preceding clauses, wherein the first substrate and the second substrate are mechanically or acoustically decoupled.

Clause 71. The blood pressure monitoring system of any of the preceding clauses, wherein the decoupling occurs due to the first substrate being separated from the second substrate.

Clause 72. The blood pressure monitoring system of any of the preceding clauses, wherein the first substrate is separated from the second substrate with a gap that is parallel to an axis along the artery.

Clause 73. The blood pressure monitoring system of any of the preceding clauses, wherein the gap is parallel to the axis but off center from the axis.

Clause 74. The blood pressure monitoring system of any of the preceding clauses, wherein the first substrate includes a portion that is perpendicular to the axis.

Clause 75. The blood pressure monitoring system of any of the preceding clauses, wherein the detector comprises a plurality of detectors.

Clause 76. The blood pressure monitoring system of any of the preceding clauses, wherein the plurality of detectors comprises 2 or more detectors.

Clause 77. The blood pressure monitoring system of any of the preceding clauses, wherein the plurality of detectors comprises 3 or more detectors.

Clause 78. The blood pressure monitoring system of any of the preceding clauses, wherein the plurality of detectors comprises 4 or more detectors.

Clause 79. The blood pressure monitoring system of any of the preceding clauses, wherein each of the plurality of detectors is coupled to a substrate arm with a substrate portion having a series of bends.

Clause 80. The blood pressure monitoring system of any of the preceding clauses, wherein each of the series of bends is 90-degrees.

Clause 81. The blood pressure monitoring system of any of the preceding clauses, wherein the plurality of detectors are aligned with an axis along the artery.

Clause 82. The blood pressure monitoring system of any of the preceding clauses, wherein, in response to the blood pressure monitoring system being in a first configuration, the exciter is centered with the detector, and in response to the blood pressure monitoring system being in a second configuration, the exciter is off center with the detector.

Clause 83. The blood pressure monitoring system of any of the preceding clauses, wherein the exciter is at a first position along the tissue of the patient above the artery, and wherein the detector is at a second position along the tissue of the patient above the artery.

Clause 84. The blood pressure monitoring system of any of the preceding clauses, wherein the first position is farther away from a wrist of the patient than the second position.

Clause 85. The blood pressure monitoring system of any of the preceding clauses, wherein the signal is an acoustic signal.

Clause 86. A physiological monitor comprising a non-transitory data store storing data collected from at least one sensor and computer-executable instructions; a processor in communication with the at least one sensor and the non-transitory data store, wherein the computer-executable instructions, when executed by the processor, configure the processor to measure blood pressure and cardiac output, wherein the blood pressure and the cardiac output are measured by using a same device.

Clause 87. The physiological monitor, wherein the physiological monitor may be the blood pressure monitoring system of any of the preceding clauses.

Clause 88. The physiological monitor of any of the preceding clauses, wherein the computer-executable instructions further include instructions, when executed by the processor, configure the processor to measure the blood pressure from a system response of a body of the patient.

Clause 89. The physiological monitor of any of the preceding clauses, wherein the computer-executable instructions further include instructions, when executed by the processor, configure the processor to obtain the system response by causing transmission of a transmit signal into the body of the patient and receiving a received signal from the patient's body.

Clause 90. The physiological monitor of any of the preceding clauses, wherein the system response is associated with a change between the transmit signal and the receive signal.

Clause 91. A blood pressure monitoring system comprising a housing comprising: a sensor interconnect portion configured to receive a sensor connector; a processor, coupled to the sensor interconnect portion, and configured to compute at least a blood pressure measurement from physiological data; and a hub interconnect portion, coupled to the processor, and configured to receive a hub connector to provide the blood pressure measurement, wherein the housing is configured to: attach to a forearm of a patient; and connect to a sensor with the sensor interconnect portion.

Clause 92. The blood pressure monitoring system of any of the preceding clauses, wherein the housing further comprises a disposable portion and a reusable portion, wherein the disposable portion includes an adhesive to attach to the forearm of the patient.

Clause 93. A blood pressure monitoring system comprising a sensor comprising a first disposable portion, and configured to attach to a forearm of a patient with the disposable portion; and a housing including: a reusable portion comprising: a processor, wherein the processor is coupled to the sensor; and a second disposable portion configured to attach to the forearm of the patient.

Clause 94. The blood pressure measurement system of any of the preceding clauses, wherein the sensor is further configured to obtain physiological measurements from a body of the patient.

Clause 95. The blood pressure monitoring system of any of the preceding clauses, wherein the processor is configured to cause transmission of a transmit signal by the sensor and receive a receive signal from the sensor, wherein the receive signal includes the physiological measurements.

Clause 96. A blood pressure monitoring system comprising: a substrate comprising: a first section; a second section, coupled to the first section, and comprising: a first portion; a second portion; a third portion; a fourth portion; and a fifth portion; a third section, coupled to the first section, and comprising: a first portion, a second portion, a third portion, and a fourth portion; and an exciter, coupled to the fourth portion of the third section, and configured to transmit a signal into tissue of a patient; and a reference detector, coupled to the third portion of the third section, and configured to receive the signal that is reflected from a body of the patient; and a detector, coupled to the third and fifth portions of the second section, and configured to receive the signal that is reflected from the body of the patient.

Clause 97. The blood pressure monitoring system of any of the preceding clause, used with any of clauses 1-93 or any other claims or clauses herein.

Clause 98. The blood pressure monitoring system of any of the preceding clauses, wherein the first portion of the third section is perpendicular to the second portion of the third section, and is parallel to the third and fourth portions of the third section.

Clause 99. The blood pressure monitoring system of any of the preceding clauses, wherein the first portion of the second section is parallel to the third and fifth portions of the second section, and is perpendicular to the second and fourth portions of the second section.

Clause 100. A magnetic exciter, configured to push a nob.

Clause 101. The magnetic exciter of Clause 100, wherein the magnetic exciter operates as the exciter of any of the above clauses.

Clause 102. The magnetic exciter of Clauses 100-101, further configured to push the nob according to a signal.

Clause 103. The magnetic exciter of Clauses 100-102, further comprising a magnet

Clause 104. The magnetic exciter of Clause 100-103, wherein the magnet includes at least two magnets

Clause 105. The magnetic exciter of Clause 100-104, wherein a first of the magnets includes a magnetic orientation opposite of a second of the two magnets

Clause 106. The magnetic exciter of Clause 100-105, further comprising a spring

Clause 107. The magnetic exciter of Clause 100-106, wherein the spring includes at least one spring

Clause 108. The magnetic exciter of Clause 100-107, wherein the spring includes at least two springs

Clause 109. The magnetic exciter of Clause 100-108, wherein the spring includes a suspension spring

Clause 110. The magnetic exciter of Clause 100-109, wherein the spring includes a coiled spring

Clause 111. The magnetic exciter of Clause 100-110, further comprising a mass, coupled to the spring, and configured to apply force to ensure the nob presses into tissue of a patient.

Clause 112. The magnetic exciter of Clause 100-111, further comprising a bobbin, coupled to the nob and the spring, configured to adjust a position to push the nob.

Clause 113. The magnetic exciter of Clause 100-112, further comprising a wire coil, coupled to the bobbin, configured to generate an electric field to adjust a position of the magnet

Clause 114. The magnetic exciter of Clause 100-113, further configured to receive a signal that causes the wire coil to generate the electric field.

Clause 115. The magnetic exciter of Clause 100-114, further comprising a wire connect, coupled to the wire coil, configured to receive a signal to apply to the wire coil.

Clause 116. A plurality of detectors, wherein the plurality of detectors are configured to obtain a signal at different times, wherein a time delay of a received signal between each of the detectors provide a variable consistent between each of the detectors to process the signal and cause computing of a blood pressure measurement.

Clause 117. The plurality of detectors of Clause 116, wherein the plurality of detectors operate as the detector of any of the above clauses.

Clause 118. The plurality of detectors of Clause 116-117, wherein a difference between the time delays for each of the received signals at each of the detectors provides information about a system response in which the plurality of detectors are placed.

Clause 119. The plurality of detectors of Clause 116-118, wherein the time delay between each of the plurality of detectors vary according to a plurality of physiological characteristics, wherein the plurality of physiological characteristics include at least one of a patient's skin characteristics, body composition, arterial placement, arterial size, and physiological parameters.

Clause 120. A method comprising: transmitting a signal into a wearer from an emitter; receiving the signal with a detector; obtaining a system response corresponding to a relation of the signal as transmitted and received; computing a blood pressure of the wearer according to the system response; and providing the blood pressure to the wearer.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain aspects, the terms “generally parallel” and “substantially parallel” refer to a value, amount, or characteristic that departs from exactly parallel by less than or equal to 10 degrees, 5 degrees, 3 degrees, or 1 degree. As another example, in certain aspects, the terms “generally perpendicular” and “substantially perpendicular” refer to a value, amount, or characteristic that departs from exactly perpendicular by less than or equal to 10 degrees, 5 degrees, 3 degrees, or 1 degree.

Many other variations than those described herein will be apparent from this disclosure. For example, certain acts, events, or functions of any of the algorithms described herein can be performed in a different sequence, can be added, merged, or left out altogether (for example, not all described acts or events are necessary for the practice of the algorithms). Moreover, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

It is to be understood that not necessarily all such advantages can be achieved in accordance with any particular example of the examples disclosed herein. Thus, the examples disclosed herein can be embodied or carried out in a manner that achieves or optimizes one advantage or group of advantages as taught herein without necessarily achieving other advantages as may be taught or suggested herein.

The various illustrative logical blocks, modules, and algorithm steps described in connection with the examples disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the examples disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry or digital logic circuitry configured to process computer-executable instructions. In another example, a processor can include an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. A computing environment can include any type of computer system, including, but not limited to, a computer system based on a microprocessor, a mainframe computer, a digital signal processor, a portable computing device, a device controller, or a computational engine within an appliance, to name a few.

The steps of a method, process, or algorithm described in connection with the examples disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

The term “substantially” when used in conjunction with the term “real-time” forms a phrase that will be readily understood by a person of ordinary skill in the art. For example, it is readily understood that such language will include speeds in which no or little delay occurs.

Conditional language used herein, such as, among others, “can,” “might,” “may,” “for example,” and the like, unless specifically stated otherwise, or otherwise understood within the context as used, is generally intended to convey that certain examples include, while other examples do not include, certain features, elements and/or states. Thus, such conditional language is not generally intended to imply that features, elements and/or states are in any way required for one or more examples or that one or more examples necessarily include logic for deciding, with or without author input or prompting, whether these features, elements and/or states are included or are to be performed in any particular example. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain examples require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C. Unless otherwise explicitly stated, the terms “set” and “collection” should generally be interpreted to include one or more described items throughout this application. Accordingly, phrases such as “a set of devices configured to” or “a collection of devices configured to” are intended to include one or more recited devices. Such one or more recited devices can also be collectively configured to carry out the stated recitations. For example, “a set of servers configured to carry out recitations A, B and C” can include a first server configured to carry out recitation A working in conjunction with a second server configured to carry out recitations B and C.

While the above detailed description has shown, described, and pointed out novel features as applied to various examples, it will be understood that various omissions, substitutions, and changes in the form and details of the devices or algorithms illustrated can be made without departing from the spirit of the disclosure. As will be recognized, the inventions described herein can be embodied within a form that does not provide all of the features and benefits set forth herein, as some features can be used or practiced separately from others.

Additionally, all publications, patents, and patent applications mentioned in this specification are herein incorporated by reference to the same extent as if each individual publication, patent, or patent application was specifically and individually indicated to be incorporated by reference.

What is claimed is:

1. A blood pressure monitoring system, comprising:
an exciter configured to produce an acoustic signal, the exciter being provided on a first substrate portion;
a detector spaced apart from the exciter, the detector being configured to detect the acoustic signal and to produce an electrical output signal, the detector being provided on a second substrate portion that is acoustically decoupled from the first substrate portion, wherein the detector comprises at least four detectors; and
a processor configured to determine a blood pressure measurement from the electrical output signal.

2. The blood pressure monitoring system of claim 1, wherein the at least four detectors are separated by at least one gap.

3. The blood pressure monitoring system of claim 1, wherein the detector comprises five detectors.

4. The blood pressure monitoring system of claim 1, further comprising a memory device.

5. The blood pressure monitoring system of claim 1, further comprising an inertial measurement unit (IMU).

6. The blood pressure monitoring system of claim 1, wherein the first substrate portion and the second substrate portion are separated by a gap.

7. The blood pressure monitoring system of claim 1, wherein the first substrate portion and the second substrate portion include acoustically absorptive material.

8. The blood pressure monitoring system of claim 1, wherein the processor is configured to determine the blood pressure measurement based on a measured phase delay between an electrical input signal and the electrical output signal.

9. The blood pressure monitoring system of claim 1, wherein the first substrate portion and the second substrate portion are flexible.

10. The blood pressure monitoring system of claim 1, further comprising a flexible circuit that connects the exciter and the detector to an electrical connector.

11. The blood pressure monitoring system of claim 1, wherein the exciter and the detector comprise a piezo device or a microelectromechanical system.

12. The blood pressure monitoring system of claim 1, further comprising an attachment element configured to attach the exciter and the detector to a forearm of a patient over a radial artery.

13. The blood pressure monitoring system of claim 12, wherein the attachment element comprises an adhesive substrate.

14. The blood pressure monitoring system of claim 12, further comprising an alignment indicator to align a measurement axis of the exciter and the detector to the radial artery.

15. The blood pressure monitoring system of claim 1, further comprising an attachment element configured to attach the exciter and the detector to a palm side of a wrist on a forearm of a patient.

16. The blood pressure monitoring system of claim 1, further comprising an attachment element configured to attach the exciter and the detector to a patient, wherein the exciter is closer to a hand of the patient than the detector.

17. The blood pressure monitoring system of claim 1, further comprising an attachment element configured to attach the exciter and the detector to a patient, wherein the detector is closer to a hand of the patient than the exciter.

18. The blood pressure monitoring system of claim 1, further comprising an attachment element configured to attach the exciter and the detector to a patient, wherein the exciter and the detector are attached parallel to a length of an arm of the patient from an elbow to a wrist of the patient.

19. The blood pressure monitoring system of claim 1, further comprising an attachment element configured to attach the exciter and the detector to a patient, wherein the exciter is positioned over a radial artery of the patient.

20. The blood pressure monitoring system of claim 1, further comprising an attachment element configured to attach the exciter and the detector to a patient, wherein the detector is positioned over a radial artery of the patient.
